(19) United States
(12) Patent Application Publication
(10) Pub. No.: US 2026/0335490 A1
(43) Pub. Date: Oct. 29, 2026

(54) FRAGRANCE COMPOSITIONS CONTAINING 1,3-BUTANEDIOL

(71) Applicant: FIRMENICH SA, Satigny (CH)

(72) Inventors: Madelyne PHAM, Joinville-le-Pont (FR); Anne-Victoire DE BAYNAST, Versailles (FR); Aude DAUGERON JOUAULT, LEVALLOIS-PERRET (FR)

(73) Assignee: FIRMENICH SA, Satigny (CH)

(21) Appl. No.: 19/104,902

(22) PCT Filed: Aug. 17, 2023
(86) PCT No.: PCT/EP2023/072636

§ 371 Date: Feb. 19, 2025

(30) Foreign Application Priority Data
Aug. 31, 2022 (EP) 22193249.4

(51) Int. Cl. A61L 9/04; A61K 8/34; A61L 101/34; A61Q 13/00; C11B 9/00

(52) U.S. Cl. CPC A61L 9/044; A61K 8/345; A61Q 13/00; C11B 9/0015; A61L 2101/34

(57) ABSTRACT

The present disclosure relates to fragrance compositions, particularly fragrance compositions comprising 1,3-butanediol. The present disclosure also relates to consumer products, such as leave-on products, for instance eau de toilette, eau de parfum, body sprays, deodorants, and the like, containing the said fragrance compositions.

### CROSS REFERENCE TO RELATED APPLICATIONS

This application claims the benefit of priority of European Application No. 22193249.4, filed Aug. 31, 2022, which is hereby incorporated by reference as though set forth herein in its entirety.

### FIELD OF THE DISCLOSURE

The present disclosure relates to fragrance compositions, particularly fragrance compositions comprising 1,3-butanediol. The present disclosure also relates to consumer products, such as leave-on products, for instance eau de toilette, eau de parfum, body sprays, deodorants, and the like, containing the said fragrance compositions.

### BACKGROUND OF THE DISCLOSURE

Controversy exists around the use of ethanol in consumer products, typically personal care products. Such controversy has arisen due to religious conviction, environmental impact, as well as a tendency towards skin irritation. Thus, a growing movement towards decreasing the use of ethanol in personal care products has been observed during the past decade. Many industries, particularly the fragrance industry, are facing a potentially new CARB regulation that seeks to limit the percentage of volatile organic compounds (VOCs) at 50% in personal fragrance products comprising ≤10% fragrance by January 2031.

However, there are challenges to reducing ethanol content in a fragrance product. One challenge is reduction of olfactive impact, which is the efficacy or intensity of a perfumery raw material during the first moments of product performance. As it is very volatile, ethanol aids in providing olfactive impact. Replacing ethanol with a solvent of lower volatility in fragrance products tends to reduce its olfactive impact, which is detrimental since impact is a very important characteristic of a fragrance as it provides the first impression about the fragrance. Another challenge is reduction of solubility of the components of a fragrance. Ethanol is useful in solubilizing fragrance components, many of which are lipophilic. Reduction of ethanol leads to reduction of solubility of fragrance components, often leading to undesirable outcomes.

Accordingly, there is an ongoing need for fragrance compositions that are low in VOC content but can elicit desirable olfactive performance as well as solubility of fragrance components.

### SUMMARY OF THE DISCLOSURE

The following aspects of the present disclosure seek to address one or more of the problems described hereinabove.

In a first aspect, the present disclosure relates to a fragrance composition comprising at least:
a) 1,3-butanediol,
b) a fragrance component, wherein the fragrance component comprises greater than 25% by weight of perfumery ingredients having a vapor pressure above 0.0008 Torr at 25 °C, relative to the weight of the fragrance component, and
c) a volatile solvent with a vapor pressure above 0.1 mmHg at 20 °C in an amount not greater than 70%, typically 20% to 70%, by weight relative to the total weight of the fragrance composition.

In a second aspect, the present disclosure relates to a consumer product comprising the fragrance composition described herein.

In a third aspect, the present disclosure relates to a method for enhancing or modulating the perceived olfactive impact and/or long-lastingness of a fragrance composition, the method comprising combining 1,3-butanediol with a fragrance component, and a volatile solvent with a vapor pressure above 0.1 mmHg at 20 °C to obtain the fragrance composition, wherein the volatile solvent with a vapor pressure above 0.1 mmHg at 20 °C is in an amount not greater than 70%, typically 20% to 70%, by weight relative to the total weight of the fragrance composition.

### BRIEF DESCRIPTION OF THE FIGURES

FIG. 1 shows a comparison of the olfactive performance between a reference composition and inventive composition, both of which contain the same fragrance.

FIG. 2 shows a comparison of the olfactive performance between inventive compositions and their corresponding control formulation.

FIG. 3 shows a comparison of the olfactive performance between inventive compositions.

FIG. 4 shows another comparison of the olfactive performance between compositions of the present disclosure.

### DETAILED DESCRIPTION

As used herein, the terms “a”, “an”, or “the” means “one or more” or “at least one” unless otherwise stated.

While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition.

Unless defined otherwise, all technical and scientific terms used herein have the same meaning as is commonly understood by one of skill in the art to which this specification pertains.

It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

Throughout the present disclosure, various chemical names and structures may be recited. Unless otherwise stated, any stereoisomers, such as enantiomers, diastereomers, anomers, epimers, and the like; and geometric isomers, such as cis/trans or E/Z isomers, of the recited chemical name or structure are contemplated. As would be understood by those of ordinary skill in the art, stereoisomers may possess one stereocenter, giving rise to enantiomers, or more than one stereocenter, giving rise to diastereomers, each stereocenter having one of two different stereochemistries (i.e., R or S). Enantiomers may be characterized by their ability to rotate oncoming plane-polarized light to the right, designated as dextrorotatory, “(+)” or “D”, or to the left, designated as levorotatory, “(−)” or “L”. Enantiomers may exist as racemic mixtures or scalemic mixtures. Geometric isomers refer to isomers in which the spatial relationship of atoms around a double bond are different, typically designated E or Z according to conventional understanding in the chemical art. Geometric isomers may also exist as mixtures of E and Z isomers. All of the aforementioned isomeric variations of the chemical names or structures recited herein are included.

In the first aspect, the present disclosure relates to a fragrance composition comprising at least:
a) 1,3-butanediol,
b) a fragrance component, wherein the fragrance component comprises greater than 25% by weight of perfumery ingredients having a vapor pressure above 0.0008 Torr at 25 °C, relative to the weight of the fragrance component, and
c) a volatile solvent with a vapor pressure above 0.1 mmHg at 20 °C in an amount not greater than 70%, typically 20% to 70%, by weight relative to the total weight of the fragrance composition.

The amount of 1,3-butanediol, also known as butylene glycol, may be present in any suitable amount. However, in some embodiments, 1,3-butanediol is present in an amount of at least 1%, typically from 1% to 50%, more typically 1% to 30%, by weight relative to the total weight of the fragrance composition. 1,3-butanediol may be synthesized according to known methods or, typically, obtained from commercial sources.

The fragrance component comprises greater than 25% by weight of perfumery ingredients having a vapor pressure above 0.0008 Torr at 25 °C, relative to the weight of the fragrance component.

In some embodiments, the fragrance component comprises greater than 45%, typically greater than 50%, more typically greater than 55%, by weight of perfumery ingredients having a vapor pressure above 0.0008 Torr at 25 °C, relative to the weight of the fragrance component.

In other embodiments, the fragrance component comprises 45% to 99%, typically 55% to 99%, more typically 60% to 99%, by weight of perfumery ingredients having a vapor pressure above 0.0008 Torr at 25 °C, relative to the weight of the fragrance component.

Perfumery ingredients having a vapor pressure above 0.0008 Torr at 25 °C may be selected from perfumery ingredients known those of ordinary skill in the art. As used herein, the term “vapor pressure” means the partial pressure in air at a defined temperature for a given chemical species. It defines a chemical species' desire to be in the gas phase rather than the liquid or solid state. The higher the vapor pressure, the greater the proportion of the material that will, at equilibrium, be found in a closed headspace. It is also related to the rate of evaporation of a perfuming ingredient which is defined in an open environment where material is leaving the system. The vapor pressure of perfumery ingredients may be determined by any method known those of ordinary skill in the art, such as methods disclosed in International Patent Application Publication No. WO 2015/089246 A1.

In one suitable method, to determine the vapor pressure for fragrance materials, one may go to the website “https://scifinder.cas.org/scifinder/view/scifinder/scifinderExplore.jsf” and follow the listed steps to acquire the vapor pressure:
1. Input the CAS registry number for the particular fragrance material.
2. Select the vapor pressure from the search results.
3. Record the vapor pressure (given in Torr at 25 °C).

SciFinder uses Advanced Chemistry Development (ACD/Labs) Software Version 11.02. (© 1994-2013). If the CAS number for the particular fragrance material is unknown or does not exist, one may utilize the ACD/Labs reference program to directly determine the vapor pressure.

Exemplary perfumery ingredients having a vapor pressure above 0.0008 Torr at 25 °C include, but are not limited, to the ingredients listed below:

| | | | | Vapor |
|---|---|---|---|---|
| | | | | Pressure |
| | CAS | | | (Torr at |
| No. | Number | Chemical Name | Common Name** | 25 °C)* |
| 1 | 139504- | 1-[[2-(1,1- | Amber core | 0.000803 |
| | 68-0 | dimethylethyl)cyclohexyl]oxy]-2- | | |
| | | butanol; | | |
| 2 | 28940- | 7-methyl-2H-1,5-benzodioxepin- | Calone | 0.000831 |
| | 11-6 | 3(4H)-one; | | |
| 3 | 72403- | 4-(4-methyl-3-penten-1-yl)-3- | Myraldylacetate | 0.000879 |
| | 67-9 | Cyclohexene-1-methanol-1- | | |
| | | acetate; | | |
| 4 | 121-32- | 3-ethoxy-4-hydroxybenzaldehyde; | Ethyl vanillin | 0.000884 |
| | 4 | | | |
| 5 | 198404- | 1-methyl-2-[(1,2,2- | Javanol ® | 0.000902 |
| | 98-7 | trimethylbicyclo[3.1.0]hex-3- | | |
| | | yl)methyl]cyclopropanemethanol; | | |
| 6 | 67633- | 1-hydroxy-3-decanone; | Methyl Lavender | 0.000951 |
| | 95-8 | | Ketone | |
| 7 | 93-08-3 | 1-(2-naphthalenyl)ethanone; | Methyl beta- | 0.000957 |
| | | | naphthyl | |
| | | | ketone | |
| 8 | 28219- | 2-methyl-4-(2,2,3-trimethyl-3- | Hindinol | 0.000961 |
| | 60-5 | cyclopenten-1-yl)-2-buten-1-ol; | | |
| 9 | 1211- | 3-oxo-2-(2Z)-2-penten-1-yl- | Methyl jasmonate | 0.000965 |
| | 29-6 | (1R,2R)-cyclopentaneacetic acid | | |
| | | methyl ester; | | |
| 10 | 109-42- | 10-undecenoic acid butyl ester; | Butyl | 0.00104 |
| | 2 | | Undecylenate | |
| 11 | 5471- | 4-(4-hydroxyphenyl)-2-butanone; | Raspberry ketone | 0.00106 |
| | 51-2 | | | |
| 12 | 106-29- | Butanoic acid (2E)-3,7-dimethyl- | Geranyl Butyrate | 0.00116 |
| | 6 | 2,6-octadien-1-yl ester; | | |
| 13 | 68039- | 2,2-dimethylpropanoic acid | Pivacyclene | 0.00119 |
| | 44-1 | 3a,4,5,6,7,7a-hexahydro-4,7- | | |
| | | methano-1H-inden-6-yl ester; | | |
| 14 | 68901- | 2-[6-methyl-8-(1- | Glycolierral | 0.00121 |
| | 32-6 | methylethyl)bicyclo[2.2.2]oct-5- | | |
| | | en-2-yl]-1,3-dioxolane; | | |
| 15 | 91-64-5 | 2H-1-Benzopyran-2-one; | Coumarin | 0.0013 |
| 16 | 107-74- | 1,7-Octanediol, 3,7-dimethyl- | Hydroxyol | 0.00139 |
| | 4 | | | |
| 17 | 41199- | 2-Naphthalenol, decahydro-2,5,5- | Ambrinol | 0.0014 |
| | 20-6 | trimethyl- | | |
| 18 | 12262- | ndecanoic acid, 3-methylbutyl | Iso Amyl | 0.0014 |
| | 03-2 | ester | Undecylenate | |
| 19 | 2050- | Benzoic acid, 2-hydroxy-, pentyl | Amyl Salicylate | 0.00144 |
| | 08-0 | ester | | |
| 20 | 3155- | 2-Butenal, 2-methyl-4-(2,6,6- | Boronal | 0.00147 |
| | 71-3 | trimethyl-1-cyclohexen-1-yl)- | | |
| 21 | 72089- | 3-Cyclopentene-1-butanol, | Brahmanol | 0.00154 |
| | 08-8 | β,2,2,3-tetramethyl-2-Methyl-4- | | |
| | | (2,2,3-trimethyl-3-cyclopenten-1- | | |
| | | yl)butanol | | |
| 22 | 3943- | Benzoic acid, 4-hydroxy-3- | Carnaline | 0.00157 |
| | 74-6 | methoxy-, methyl ester | | |
| 23 | 55418- | 2-Butanone, 4-(1,3-benzodioxol- | Dulcinyl | 0.00161 |
| | 52-5 | 5-yl)- | | |
| 24 | 75490- | Benzenebutanenitrile, α,α, γ- | Khusinil | 0.00162 |
| | 39-0 | trimethyl- | | |
| 25 | 40785- | Cyclododeca[c]furan, | Muscogene | 0.00163 |
| | 62-4 | 1,3,3a,4,5,6,7,8,9,10,11,13a- | | |
| | | dodecahydro- | | |
| 26 | 10094- | Butanoic acid, 1,1-dimethyl-2- | Dimethyl Benzyl | 0.00168 |
| | 34-5 | phenylethylester | Carbinyl Butyrate | |
| 27 | 285977- | (2,5-Dimethyl-1,3-dihydroinden-2- | Lilyflore | 0.0018 |
| | 85-7 | yl)methanol | | |
| 28 | 211299- | 4H-4a,9-Methanoazuleno[5,6-d]- | Ambrocenide | 0.00182 |
| | 54-6 | 1,3-dioxole, octahydro- | | |
| | | 2,2,5,8,8,9a-hexamethyl-, | | |
| | | (4aR,5R,7aS,9R)- | | |
| 29 | 76842- | 4,7-Methano-1H-inden-6-ol, | Frutene | 0.00184 |
| | 49-4 | 3a,4,5,6,7,7a-hexahydro-8,8- | | |
| | | dimethyl-,6-propanoate | | |
| 30 | 121-39- | 2-Oxiranecarboxylic acid,3- | Ethyl Phenyl | 0.00184 |
| | 1 | phenyl-, ethyl ester | Glycidate | |
| 31 | 77-54-3 | 1H-3a,7-Methanoazulen-6-ol, | Cedac | 0.00192 |
| | | octahydro-3,6,8,8-tetramethyl-, 6- | | |
| | | acetate,(3R,3aS,6R,7R,8aS)- | | |
| 32 | 121-33- | Benzaldehyde, 4-hydroxy-3- | Vanillin | 0.00194 |
| | 5 | methoxy- | | |
| 33 | 406488- | 2-Ethyl-N-methyl-N-(m- | Paradisamide | 0.002 |
| | 30-0 | tolyl)butanamide | | |
| 34 | 25152- | 3-Hexen-1-ol, 1-benzoate,(3Z)- | Cis-3-Hexenyl | 0.00203 |
| | 85-6 | | Benzoate | |
| 35 | 13215- | 2-Cyclohexen-1-one, 4-(2-buten- | Tabanone Coeur | 0.00223 |
| | 88-8 | 1-ylidene)-3,5,5-trimethyl- | | |
| 36 | 103694- | Benzenepropanol, β,β,3-trimethyl- | Majantol | 0.00224 |
| | 68-4 | | | |
| 37 | 122-40- | Heptanal, 2-(phenylmethylene)- | Amyl Cinnamic | 0.00233 |
| | 7 | | Aldehyde | |
| 38 | 65405- | 1-Naphthalenol, 1,2,3,4,4a,7,8,8a- | Oxyoctaline | 0.00236 |
| | 72-3 | octahydro-2,4a,5,8a-tetramethyl-, | Formate | |
| | | 1-formate | | |
| 39 | 67634- | Propanoic acid, 2-methyl-, | Cyclabute | 0.00244 |
| | 20-2 | 3a,4,5,6,7,7a-hexahydro-4,7- | | |
| | | methano-1H-inden-5-yl ester | | |
| 40 | 27606- | Indeno[1,2-d]-1,3- | Magnolan | 0.00251 |
| | 09-3 | dioxin,4,4a,5,9b-tetrahydro-2,4- | | |
| | | dimethyl- | | |
| 41 | 141-13- | 9-Undecenal, 2,6,10-trimethyl- | Adoxal | 0.00257 |
| | 9 | | | |
| 42 | 2110- | Pyridine, 2-(3-phenylpropyl)- | Corps Racine VS | 0.00257 |
| | 18-1 | | | |
| 43 | 97384- | Benzenepropanenitrile, α-ethenyl- | Citrowanil B | 0.00265 |
| | 48-0 | α-methyl- | | |
| 44 | 33704- | 4H-Inden-4-one, 1,2,3,5,6,7- | Cashmeran | 0.00269 |
| | 61-9 | hexahydro-1,1,2,3,3-pentamethyl- | | |
| 45 | 36306- | Cyclohexanone, 4-(1- | Kephalis | 0.00269 |
| | 87-3 | ethoxyethenyl)-3,3,5,5- | | |
| | | tetramethyl- | | |
| 46 | 1205- | 1,3-Benzodioxole-5-propanal, α- | Helional | 0.0027 |
| | 17-0 | methyl- | | |
| 47 | 104-67- | 2(3H)-Furanone, 5-heptyldihydro- | gamma- | 0.00271 |
| | 6 | | Undecalactone | |
| | | | (racemic) | |
| 48 | 7388- | 3-Buten-2-one, 4-(2,2-dimethyl-6- | lonone Gamma | 0.00272 |
| | 22-9 | methylenecyclohexyl)-3-methyl- | Methyl | |
| 49 | 72903- | 1,4-Cyclohexanedicarboxylic acid, | Fructalate ™ | 0.00274 |
| | 27-6 | 1,4-diethyl ester | | |
| 50 | 127-51- | 3-Buten-2-one, 3-methyl-4-(2,6,6- | Isoraldeine Pure | 0.00282 |
| | 5 | trimethyl-2-cyclohexen-1-yl)- | | |
| | | cyclohexen-1-yl)- | | |
| 51 | 7779- | 1-Penten-3-one, 1-(2,6,6- | Methyl lonone | 0.00286 |
| | 30-8 | trimethyl-2-cyclohexen-1-yl)- | | |
| 52 | 137-00- | 5-Thiazoleethanol, 4-methyl- | Sulfurol | 0.00297 |
| | 8 | methyl- | | |
| 53 | 313973- | 1,6-Heptadien-3-one, 2- | Pharaone | 0.00298 |
| | 37-4 | cyclohexyl- | | |
| 54 | 68611- | Naphtho[2,1-b]furan, 9b- | Grisalva | 0.00305 |
| | 23-4 | ethyldodecahydro-3a,7,7- | | |
| | | trimethyl- | | |
| 55 | 107-75- | Octanal, 7-hydroxy-3,7-dimethyl- | Hydroxycitronellal | 0.00318 |
| | 5 | | | |
| 56 | 476332- | 2H-Indeno[4,5-b]furan,decahydro- | Amber Xtreme | 0.00323 |
| | 65-7 | 2,2,6,6,7,8,8-heptamethyl- | Compound 1 | |
| 57 | 68901- | Acetic acid, 2-(cyclohexyloxy)-, 2- | Cyclogalbanate | 0.00323 |
| | 15-5 | propen-1-yl ester | | |
| 58 | 93-29-8 | Phenol, 2-methoxy-4-(1-propen-1- | Iso Eugenol | 0.00324 |
| | | yl)-, 1-acetate | Acetate | |
| 59 | 7070- | Ethanol, 2-[(1R,2R,4R)-1,7,7- | Arbanol | 0.00326 |
| | 15-7 | trimethylbicyclo[2.2.1]hept-2- | | |
| | | yl]oxy]-, rel- | | |
| 60 | 41816- | Spiro[1,4-methanonaphthalene- | Rhubofix | 0.00332 |
| | 03-9 | 2(1H),2′-oxirane], 3,4,4a,5,8,8a- | | |
| | | hexahydro-3′,7-dimethyl- | | |
| 61 | 105-90- | 2,6-Octadien-1-ol, 3,7-dimethyl-, | Geranyl | 0.00336 |
| | 8 | 1-propanoate, (2E)- | Propionate | |
| 62 | 140-26- | Butanoic acid, 3-methyl-,2- | Beta Phenyl Ethyl | 0.00347 |
| | 1 | phenylethyl ester | Isovalerate | |
| 63 | 78417- | 2,4,7-Decatrienoic acid, ethyl ester | Ethyl 2,4,7- | 0.00353 |
| | 28-4 | | decatrienoate | |
| 64 | 32764- | 2H-Pyran-2-one, tetrahydro-6-(3- | Jasmolactone | 0.00355 |
| | 98-0 | penten-1-yl)- | | |
| 65 | 80858- | Benzene, [2- | Phenafleur | 0.0038 |
| | 47-5 | (cyclohexyloxy)ethyl]- | | |
| 66 | 52474- | 3-Cyclohexene-1- | Precyclemone B | 0.00381 |
| | 60-9 | carboxaldehyde, 1-methyl-3-(4- | | |
| | | methyl-3-penten-1-yl)- | | |
| 67 | 139539- | 6-Oxabicyclo[3.2.1]octane, 5- | Cassifix | 0.00381 |
| | 66-5 | methyl-1-(2,2,3-trimethyl-3- | | |
| | | cyclopenten-1-yl)- | | |
| 68 | 23911- | Ethanone, 1-(3-methyl-2- | Nerolione | 0.00383 |
| | 56-0 | benzofuranyl)- | | |
| 69 | 173445- | 1H-Indene-5-propanal, 2,3- | Hivernal (A- | 0.00392 |
| | 65- | dihydro-3,3-dimethyl- | isomer) | |
| 70 | 87731- | Carbonic acid, 4-cycloocten-1-yl | Violiff | 0.00401 |
| | 18-8 | methylester | | |
| 71 | 65442- | Quinoline, 6-(1-methylpropyl)- | Iso Butyl Quinoline | 0.00408 |
| | 31-1 | | | |
| 72 | 79-69-6 | 3-Buten-2-one, 4-(2,5,6,6- | alpha-Irone | 0.00419 |
| | | tetramethyl-2-cyclohexen-1-yl)- | | |
| 73 | 94400- | Naphth[2,3- | Molaxone | 0.00425 |
| | 98-3 | bloxirene, 1a,2,3,4,5,6,7,7a- | | |
| | | octahydro-1a,3,3,4,6,6- | | |
| | | hexamethyl-,(1aR,4S,7aS)-rel- | | |
| 74 | 58567- | Cyclododecane, (ethoxymethoxy)- | Boisambren Forte | 0.00433 |
| | 11-6 | | | |
| 75 | 18871- | Pentitol, 1,5-anhydro-2,4-dideoxy- | Jasmal | 0.00434 |
| | 14-2 | 2-pentyl-, 3-acetate | | |
| 76 | 68991- | 2-Naphthalenecarboxaldehyde, | Melafleur | 0.00436 |
| | 97-9 | 1,2,3,4,5,6,7,8-octahydro-8,8- | | |
| | | dimethyl- | | |
| 77 | 86803- | 4,7-Methano-1H-indene-2- | Scentenal | 0.00439 |
| | 90-9 | carboxaldehyde,octahydro-5- | | |
| | | methoxy- | | |
| 78 | 80-54-6 | 4-(1,1-dimethylethyl)-α- | Lilial | 0.00444 |
| | | methylbenzenepropanal | | |
| 79 | 7493- | Acetic acid, 2-phenoxy-,2-propen- | Allyl Phenoxy | 0.00454 |
| | 74-5 | 1-yl ester | Acetate | |
| 80 | 55066- | Benzenepentanal, β-methyl- | Mefranal | 0.00455 |
| | 49-4 | | | |
| 81 | 54992- | 3-Buten-2-one, 4-(2,2,3,6- | Myrrhone | 0.0046 |
| | 90-4 | tetramethylcyclohexyl)- | | |
| 82 | 141-14- | 6-Octen-1-ol, 3,7-dimethyl-, 1- | Citronellyl | 0.00469 |
| | 0 | propanoate | Propionate | |
| 83 | 2437- | Dodecanenitrile | Clonal | 0.0048 |
| | 25-4 | | | |
| 84 | 80-71-7 | 2-Cyclopenten-1-one, 2-hydroxy- | Maple Lactone | 0.00484 |
| | | 3-methyl- | | |
| 85 | 67662- | Propanoic acid, 2,2-dimethyl-, 2- | Pivarose Q | 0.00484 |
| | 96-8 | phenylethylester | | |
| 86 | 23696- | 2-Buten-1-one, 1-(2,6,6-trimethyl- | Damascenone | 0.00503 |
| | 85-7 | 1,3-cyclohexadien-1-yl)- | | |
| 87 | 67663- | 2(3H)-Furanone, 5-hexyldihydro- | Peacholide | 0.00512 |
| | 01-8 | 4-methyl-tetramethyl- | | |
| 88 | 97-54-1 | Phenol, 2-methoxy-4-(1-propen-1- | Isoeugenol | 0.00519 |
| | | yl)- | | |
| 89 | 10339- | 1,6-Nonadien-3-ol, 3,7-dimethyl- | Ethyl linalool | 0.0052 |
| | 55-6 | | | |
| 90 | 23267- | 3-Buten-2-one, 4-(2,2,6-trimethyl- | lonone Epoxide | 0.0052 |
| | 57-4 | 7-oxabicyclo[4.1.0]hept-1-yl)- | Beta | |
| 91 | 171102- | 4,7-Methano-1H-inden-6-ol, | Flor Acetate | 0.0053 |
| | 41-3 | 3a,4,5,6,7,7a-hexahydro-8,8- | | |
| | | dimethyl-, 6-acetate | | |
| 92 | 65405- | Cyclohexanebutanal, α,2,6,6- | Cetonal ® | 0.00533 |
| | 84-7 | tetramethyl- | | |
| 93 | 18096- | Indeno[1,2-d]-1,3-dioxin, | Indoflor ® | 0.00557 |
| | 62-3 | 4,4a,5,9b-tetrahydro- | | |
| 94 | 63500- | 2H-Pyran-4-ol, tetrahydro-4- | Florosa Q/Florol | 0.00557 |
| | 71-0 | methyl-2-(2-methylpropyl)- | | |
| 95 | 103-60- | Propanoic acid, 2-methyl-, 2- | Phenoxy Ethyl iso- | 0.00562 |
| | 6 | phenoxyethyl ester | Butyrate | |
| 96 | 37677- | 3-Cyclohexene-1- | Iso Hexenyl | 0.00565 |
| | 14-8 | carboxaldehyde, 4-(4-methyl-3- | Cyclohexenyl | |
| | | penten-1-yl)- | Carboxaldehyde | |
| 97 | 77-83-8 | 2-Oxiranecarboxylic acid, 3- | Ethyl Methyl | 0.00571 |
| | | methyl-3-phenyl-, ethyl ester | Phenyl Glycidate | |
| 98 | 38462- | Cyclohexanone, 2-(1-mercapto-1- | Ringonol 50 TEC | 0.00585 |
| | 22-5 | methylethyl)-5-methyl- | | |
| 99 | 4430- | 2H-1-Benzopyran-2-one, | Octahydro | 0.00586 |
| | 31-3 | octahydro- | Coumarin | |
| 100 | 87-19-4 | Benzoic acid, 2-hydroxy-, 2- | Iso Butyl Salicylate | 0.00613 |
| | | methylpropyl ester | | |
| 101 | 2785- | Phenol, 2-methoxy-4-propyl- | Dihydro Eugenol | 0.00624 |
| | 87-7 | | | |
| 102 | 495-62- | Cyclohexene, 4-(1,5-dimethyl-4- | Bisabolene | 0.0063 |
| | 5 | hexen-1-ylidene)-1-methyl- | | |
| 103 | 25634- | 2-Methyl-5-phenylpentan-1-ol | Rosaphen ® | 0.00637 |
| | 93-9 | | 600064 | |
| 104 | 55066- | 3-Methyl-5-phenylpentanol | Phenyl Hexanol | 0.00637 |
| | 48-3 | | | |
| 105 | 75147- | Bicyclo[3.2.1]octan-8-one,1,5- | Buccoxime | 0.00647 |
| | 23-8 | dimethyl-, oxime | | |
| 106 | 41724- | 1,4-Methanonaphthalen-6(2H)- | Plicatone | 0.00652 |
| | 19-0 | one, octahydro-7-methyl- | | |
| 107 | 35044- | 2-Buten-1-one, 1-(2,6,6-trimethyl- | beta-Damascone | 0.00655 |
| | 68-9 | 1-cyclohexen-1-yl)- | | |
| 108 | 3288- | Benzeneacetonitrile, 4-(1,1- | Marenil CI | 0.00665 |
| | 99-1 | dimethylethyl)- | | |
| 109 | 63767- | Cyclohexanemethanol, Î±-methyl- | Mugetanol | 0.00678 |
| | 86-2 | 4-(1-methylethyl)- | | |
| 110 | 92015- | 2(3H)-Benzofuranone, hexahydro- | Natactone | 0.0068 |
| | 65-1 | 3,6-dimethyl- | | |
| 111 | 33885- | Bicyclo[3.1.1]hept-2-ene-2- | Pinyl Iso Butyrate | 0.00685 |
| | 52-8 | propanal, α,α,6,6-tetramethyl- | Alpha | |
| 112 | 42604- | Cyclododecane, | Boisambrene | 0.00686 |
| | 12-6 | (methoxymethoxy)- | | |
| 113 | 5182- | 1,3-Dioxane, 2,4,6-trimethyl-4- | Floropal | 0.00709 |
| | 36-5 | phenyl- | | |
| 114 | 103-36- | 2-Propenoic acid, 3-phenyl-, ethyl | Ethyl Cinnamate | 0.00729 |
| | 6 | ester | | |
| 115 | 128-51- | Bicyclo[3.1.1]hept-2-ene-2- | Nopyl Acetate | 0.00751 |
| | 8 | ethanol, 6,6-dimethyl-,2-acetate | | |
| 116 | 56973- | 4-Penten-1-one, 1-(5,5-dimethyl- | Neobutenone α | 0.00763 |
| | 85-4 | 1-cyclohexen-1-yl)- | | |
| 117 | 40527- | 1,3-Benzodioxole, 5- | Heliotropin Diethyl | 0.00796 |
| | 42-2 | (diethoxymethyl)- | Acetal | |
| 118 | 67634- | Benzenepropanal, 4-ethyl-α,α- | Floralozone | 0.00808 |
| | 15-5 | dimethyl- | | |
| 119 | 705-86- | 2H-Pyran-2-one,tetrahydro-6- | Decalactone | 0.00825 |
| | 2 | pentyl- | | |
| 120 | 24720- | 2-Buten-1-one, 1-(2,6,6-trimethyl- | α-Damascone | 0.0083 |
| | 09-0 | 2-cyclohexen-1-yl)-, (2E)- | | |
| 121 | 39872- | 2-Buten-1-one, 1-(2,4,4-trimethyl- | Isodamascone | 0.0083 |
| | 57-6 | 2-cyclohexen-1-yl)-, (2E)- | | |
| 122 | 706-14- | 2(3H)-Furanone, 5-hexyldihydro- | γ-Decalactone | 0.00852 |
| | 9 | | | |
| 123 | 104-61- | 2(3H)-Furanone, dihydro-5-pentyl- | γ-Nonalactone | 0.00858 |
| | 0 | | | |
| 124 | 94201- | 1-Oxaspiro[4.5]decan-2-one, 8- | Methyl Laitone | 0.00872 |
| | 19-1 | methyl- | 10% TEC | |
| 125 | 13019- | 9-Decen-1-ol | Rosalva | 0.00879 |
| | 22-2 | | | |
| 126 | 103-95- | Benzenepropanal, α-methyl-4-(1- | Cymal | 0.00881 |
| | 7 | methylethyl)- | | |
| 127 | 692-86- | 10-Undecenoic acid, ethyl ester | Ethyl | 0.00882 |
| | 4 | | Undecylenate | |
| 128 | 61792- | 2,6-Nonadienenitrile, 3,7- | Lemonile | 0.00884 |
| | 11-8 | dimethyl- | | |
| 129 | 7011- | 2(3H)-Furanone, 5-hexyldihydro- | Lactojasmone | 0.00885 |
| | 83-8 | 5-methyl- | | |
| 130 | 68738- | 2-Naphthalenecarboxaldehyde, | Cyclomyral | 0.0092 |
| | 94-3 | octahydro-8,8-dimethyl | | |
| 131 | 2705- | Cyclohexanepropanoic acid, 2- | Allyl Cyclohexane | 0.00925 |
| | 87-5 | propen-1-yl ester | Propionate | |
| 132 | 86-26-0 | 1,1′-Biphenyl, 2-methoxy- | Methyl Diphenyl | 0.00928 |
| | | | Ether | |
| 133 | 6790- | (3aR,5aS,9aS,9bR)-3a,6,6,9a- | Ambronat or | 0.0093 |
| | 58-5 | tetramethyl-2,4,5,5a,7,8,9,9b- | Ambroxan | |
| | | octahydro-1H- | | |
| | | benzo[e][1]benzofuran | | |
| 134 | 148-05- | Dodecanoic acid, 12-hydroxy-, λ- | Dodecalactone | 0.00931 |
| | 1 | lactone (6CI,7CI); 1,12- | | |
| 135 | 51519- | 1,4-Methanonaphthalen-5(1H)- | Tamisone | 0.00932 |
| | 65-4 | one, 4,4a,6,7,8,8a-hexahydro- | | |
| 136 | 18127- | Benzenepropanal, 4-(1,1- | Bourgeonal | 0.00934 |
| | 01-0 | dimethylethyl)- | | |
| 137 | 3738- | Naphtho[2,1- | Ambrox or Cetalox | 0.00934 |
| | 00-9 | b]furan,dodecahydro-3a,6,6,9a- | or Synambran | |
| | | tetramethyl- | | |
| 138 | 103-54- | 2-Propen-1-ol, 3-phenyl-,1- | Cinnamyl Acetate | 0.0094 |
| | 8 | acetate | | |
| 139 | 3025- | 2,4-Decadienoic acid,ethyl ester, | Ethyl 2,4- | 0.00954 |
| | 30-7 | (2E,4Z)- | Decadienoate | |
| 140 | 68683- | 1-Cyclohexene-1-ethanol,4-(1- | Iso Bergamate | 0.00965 |
| | 20-5 | methylethyl)-, 1-formate | | |
| 141 | 7492- | Acetaldehyde, 2-[(3,7-dimethyl-6- | Citronellyloxyaceta | 0.00967 |
| | 67-3 | octen-1-yl)oxy]- | Idehyde | |
| 142 | 488-10- | 2-Cyclopenten-1-one, 3-methyl-2- | Cis Jasmone | 0.00982 |
| | 8 | (2Z)-2-penten-1-yl- | | |
| 143 | 1885- | 2-Propenenitrile, 3-phenyl-, (2E)- | Cinnamalva | 0.0101 |
| | 38-7 | | | |
| 144 | 4826- | 2-Dodecenal | 2 Dodecene-1-al | 0.0102 |
| | 62-4 | | | |
| 145 | 20407- | 2-Dodecenal, (2E)- | Aldehyde | 0.0102 |
| | 84-5 | | Mandarin | |
| 146 | 5462- | Benzenepropanal, 4-methoxy-α- | Canthoxal | 0.0102 |
| | 06-6 | methyl- | | |
| 147 | 94-60-0 | 1,4-Cyclohexanedicarboxylic acid, | Dimethyl 1,4- | 0.0102 |
| | | 1,4-dimethyl ester | cyclohexanedicarb | |
| | | | oxylate | |
| 148 | 57378- | 2-Buten-1-one, 1-(2,6,6-trimethyl- | delta-Damascone | 0.0102 |
| | 68-4 | 3-cyclohexen-1-yl)- | | |
| 149 | 17283- | 2-Butanone, 4-(2,6,6-trimethyl-1- | Dihydro Beta | 0.0102 |
| | 81-7 | cyclohexen-1-yl)- | lonone | |
| 150 | 97-53-0 | Phenol, 2-methoxy-4-(2-propen-1- | Eugenol | 0.0104 |
| | | yl)- | | |
| 151 | 120-57- | 1,3-Benzodioxole-5- | Heliotropin | 0.0104 |
| | 0 | carboxaldehyde | | |
| 152 | 93-04-9 | Naphthalene, 2-methoxy- | Beta Naphthyl | 0.0104 |
| | | | Methyl | |
| | | | Ether Extra 99 | |
| 153 | 67845- | Bicyclo[2.2.2]oct-5-ene-2- | Maceal | 0.0106 |
| | 30-1 | carboxaldehyde, 6-methyl-8-(1- | | |
| | | methylethyl)- | | |
| 154 | 81782- | 3-Decen-5-ol, 4-methyl- | 4-Methyl-3-decen- | 0.0107 |
| | 77-6 | | 5-ol | |
| 155 | 5454- | Propanoic acid, decyl ester | N-Decyl | 0.011 |
| | 19-3 | | Propionate | |
| 156 | 93-16-3 | Benzene, 1,2-dimethoxy-4-(1- | Methyl Iso | 0.011 |
| | | propen-1-yl)- | Eugenol | |
| 157 | 67634- | Benzenepropanal, 2-ethyl-α,α- | Florazon (ortho- | 0.0111 |
| | 14-4 | dimethyl- | isomer) | |
| 158 | 103-26- | 2-Propenoic acid, 3-phenyl-, | Methyl Cinnamate | 0.0112 |
| | 4 | methyl ester | | |
| 159 | 23495- | Ethanol, 2-phenoxy-, 1- | Phenoxy Ethyl | 0.0113 |
| | 12-7 | propanoate | Propionate | |
| 160 | 104-54- | 2-Propen-1-ol, 3-phenyl- | Cinnamic Alcohol | 0.0117 |
| | 1 | | | |
| 161 | 78-35-3 | Propanoic acid, 2-methyl-, 1- | Linalyl Isobutyrate | 0.0117 |
| | | ethenyl-1,5-dimethyl-4-hexen-1-yl | | |
| | | ester | | |
| 162 | 116044- | Bicyclo[2.2.1]hept-5-ene-2- | Herbanate | 0.0121 |
| | 44-1 | carboxylic acid, 3-(1-methylethyl)-, | | |
| | | ethyl ester, (1R,2S,3S,4S)-rel- | | |
| 163 | 65-85-0 | Benzoic Acid | Benzoic Acid | 0.0122 |
| 164 | 61444- | 3-Hexenoic acid, (3Z)-3-hexen-1- | cis-3-hexenyl-cis- | 0.0122 |
| | 38-0 | yl ester, (3Z)- | 3-hexenoate | |
| 165 | 71077- | 4,9-Decadienal, 4,8-dimethyl- | Floral Super | 0.0123 |
| | 31-1 | | | |
| 166 | 110-98- | 2-Propanol, 1,1′-oxybis- | Dipropylene Glycol | 0.0125 |
| | 5 | | | |
| 167 | 41890- | 2-Octanol, 7-methoxy-3,7- | Osyrol ® | 0.0125 |
| | 92-0 | dimethyl- | | |
| 168 | 54440- | 1H-Inden-1-one, 2,3-dihydro- | Safraleine | 0.0126 |
| | 17-4 | 2,3,3-trimethyl- | | |
| 169 | 75975- | Bicyclo[7.2.0]undec-4-ene, | Vetyvenal | 0.0128 |
| | 83-6 | 4,11,11-trimethyl-8-methylene-, | | |
| | | (1R,4E,9S)- | | |
| 170 | 19870- | 1H-3a,7- | Cedryl methyl | 0.0128 |
| | 74-7 | Methanoazulene, octahydro-6- | ether | |
| | | methoxy-3,6,8,8-tetramethyl-, | | |
| | | (3R,3aS,6S,7R,8aS)- | | |
| 171 | 87-44-5 | Bicyclo[7.2.0]undec-4-ene, | Caryophyllene | 0.0128 |
| | | 4,11,11-trimethyl-8-methylene-, | Extra | |
| | | (1R,4E,9S)- | | |
| 172 | 106-24- | 2,6-Octadien-1-ol, 3,7-dimethyl-, | Geraniol | 0.0133 |
| | 1 | (2E)- | | |
| 173 | 106-25- | 2,6-Octadien-1-ol, 3,7-dimethyl-, | Nerol | 0.0133 |
| | 2 | (2Z)- | | |
| 174 | 30310- | 2H-Pyran, tetrahydro-2-methyl-4- | Pelargene | 0.0135 |
| | 41-9 | methylene-6-phenyl- | | |
| 175 | 68845- | Bicyclo[3.3.1 ]nonane, 2-ethoxy- | Boisiris | 0.0135 |
| | 00-1 | 2,6,6-trimethyl-9-methylene- | | |
| 176 | 2500- | 4,7-Methano-1H-inden-5-ol, | Flor Acetate | 0.0137 |
| | 83-6 | 3a,4,5,6,7,7a-hexahydro-, 5- | | |
| | | acetate | | |
| 177 | 150-84- | 6-Octen-1-ol, 3,7-dimethyl-, 1- | Citronellyl acetate | 0.0137 |
| | 5 | acetate | | |
| 178 | 151-05- | Benzeneethanol, α,α-dimethyl-, 1- | Dimethyl Benzyl | 0.0139 |
| | 3 | acetate | CarbinylAcetate | |
| 179 | 127-41- | 3-Buten-2-one, 4-(2,6,6-trimethyl- | Alpha-lonone | 0.0144 |
| | 3 | 2-cyclohexen-1-yl)-, (3E)- | | |
| 180 | 154171- | Spiro[1,3-dioxolane-2,8′(5′H)-[2H- | Ysamber ® K | 0.0147 |
| | 77-4 | 2,4a]methanonaphthalene],hexah | | |
| | | ydro-1′,1′,5′,5′-tetramethyl-, | | |
| | | (2′S,4′aS,8′aS)- (9CI) | | |
| 181 | 154171- | Spiro[1,3-dioxolane-2,8′(5′H)-[2H- | Ysamber | 0.0147 |
| | 76-3 | 2,4a]methanonaphthalene],hexahydro- | | |
| | | 1′,1′,5′,5′-tetramethyl- | | |
| 182 | 105-86- | 2,6-Octadien-1-ol, 3,7-dimethyl-, | Geranyl Formate | 0.0154 |
| | 2 | 1-formate, (2E)- | | |
| 183 | 100-06- | Ethanone, 1-(4-methoxyphenyl)- | Para Methoxy | 0.0155 |
| | 1 | | Acetophenone | |
| 184 | 134-20- | Benzoic acid, 2-amino-,methyl | Methyl | 0.0158 |
| | 3 | ester | Anthranilate | |
| 185 | 95-41-0 | 2-Cyclopenten-1-one, 2-hexyl- | Iso Jasmone T | 0.016 |
| 186 | 64001- | 4,7-Methano-1H-inden-5-ol, | Dihydro Cyclacet | 0.0163 |
| | 15-6 | octahydro-, 5-acetate | | |
| 187 | 14901- | 3-Buten-2-one, 4-(2,6,6-trimethyl- | Beta-lonone | 0.0169 |
| | 07-6 | 1-cyclohexen-1-yl)-, (3E)- | | |
| 188 | 57934- | 2-Cyclohexene-1-carboxylic acid, | Givescone | 0.0171 |
| | 97-1 | 2-ethyl-6,6-dimethyl-, ethyl ester | | |
| 189 | 1209- | 5- | Tobacarol | 0.0173 |
| | 61-6 | Oxatricyclo[8.2.0.04,6]dodecane, | | |
| | | 4,9,12,12-tetramethyl- | | |
| 190 | 29214- | Octanoic acid, 2-acetyl-,ethyl | Gelsone | 0.0179 |
| | 60-6 | ester | | |
| 191 | 18479- | 4,6-Octadien-3-ol, 3,7-dimethyl- | Muguol | 0.018 |
| | 54-4 | | | |
| 192 | 543-39- | 7-Octen-2-ol, 2-methyl-6- | Myrcenol | 0.0182 |
| | 5 | methylene- | | |
| 193 | 7775- | Benzenepropanal, 4-(1- | Cyclemax | 0.0182 |
| | 00-0 | methylethyl)- | | |
| 194 | 7540- | 6-Octen-1-ol, 3,7-dimethyl-, (3S)- | L-Citronellol | 0.0183 |
| | 51-4 | | | |
| 195 | 106-22- | 6-Octen-1-ol, 3,7-dimethyl- | Citronellol | 0.0183 |
| | 9 | | | |
| 196 | 26330- | (E)-6-ethyl-3-methyloct-6-en-1-ol | Super Muguet | 0.0185 |
| | 65-4 | | | |
| 197 | 13828- | Cyclohexanemethanol, 4-(1- | Mayol | 0.0192 |
| | 37-0 | methylethyl)-, cis- | | |
| 198 | 85-91-6 | Benzoic acid, 2-(methylamino)-, | Dimethyl | 0.0193 |
| | | methylester | anthranilate | |
| 199 | 216970- | Bicyclo[4.3.1]decane, 3-methoxy- | 3-Methoxy-7,7- | 0.0196 |
| | 21-7 | 7,7-dimethyl-10-methylene- | dimethyl-10- | |
| | | | methylenebicyclo[ | |
| | | | 4.3.1]decane | |
| 200 | 141-25- | 7-Octen-1-ol, 3,7-dimethyl- | Rhodinol | 0.0197 |
| | 3 | | | |
| 201 | 7493- | Benzene, [2-(1- | Acetaldehyde | 0.0199 |
| | 57-4 | propoxyethoxy)ethyl]- | | |
| 202 | 1128- | 2-Cyclopenten-1-one, 3-methyl-2- | Dihydrojasmone | 0.0202 |
| | 08-1 | pentyl- | | |
| 203 | 2563- | Phenol, 2-ethoxy-4-methyl- | Ultravanil | 0.0203 |
| | 07-7 | | | |
| 204 | 137-03- | Cyclopentanone, 2-heptyl- | Frutalone | 0.0204 |
| | 1 | | | |
| 205 | 104-21- | Benzenemethanol, 4-methoxy-, 1- | Anisyl Acetate | 0.0205 |
| | 2 | acetate | | |
| 206 | 1365- | 2-Furanmethanol, 5- | Linalool Oxide | 0.0205 |
| | 19-1 | ethenyltetrahydro-α,α,5-trimethyl- | | |
| 207 | 125109- | Benzenepropanal, β-methyl-3-(1- | Florhydral | 0.0207 |
| | 85-5 | methylethyl)- | | |
| 208 | 83926- | Cyclohexanepropanol, α,α- | Coranol | 0.021 |
| | 73-2 | dimethyl- | | |
| 209 | 93939- | 5,8-Methano-2H-1-benzopyran, 6- | Rhuboflor | 0.0212 |
| | 86-7 | ethylideneoctahydro- | | |
| 210 | 136-60- | Benzoic acid, butyl ester | Butyl Benzoate | 0.0217 |
| | 7 | | | |
| 211 | 101-84- | Benzene, 1,1′-oxybis- | Diphenyl Oxide | 0.0223 |
| | 8 | | | |
| 212 | 30385- | 6-Octen-2-ol, 2,6-dimethyl- | Dihydromyrcenol | 0.0244 |
| | 25-2 | | | |
| 213 | 51566- | 6-Octenenitrile, 3,7-dimethyl- | Citronellyl Nitrile | 0.0247 |
| | 62-2 | | | |
| 214 | 60335- | 2H-Pyran, 3,6-dihydro-4-methyl-2- | Rosyrane Super | 0.0247 |
| | 71-9 | phenyl- | | |
| 215 | 57576- | Cyclohexanol, 5-methyl-2-(1- | Iso Pulegol | 0.0248 |
| | 09-7 | methylethenyl)-, 1-acetate, | Acetate | |
| | | (1R,2S,5R)- | | |
| 216 | 123-11- | Benzaldehyde, 4-methoxy- | Anisic Aldehyde | 0.0249 |
| | 5 | | | |
| 217 | 68141- | Undecane, 1,1-dimethoxy-2- | Methyl Nonyl | 0.0255 |
| | 17-3 | methyl- | Acetaldehyde | |
| | | | Dimethyl | |
| | | | Acetal | |
| 218 | 2206- | Benzenemethanol, α-methylene-, | Indocolore | 0.0255 |
| | 94-2 | 1-acetate | | |
| 219 | 10528- | Cyclohexanepropanol, α-methyl- | Cyclohexylmagnol | 0.0255 |
| | 67-3 | | | |
| 220 | 141-12- | 2,6-Octadien-1-ol, 3,7-dimethyl-, | Neryl Acetate | 0.0256 |
| | 8 | 1-acetate, (2Z)- | | |
| 221 | 105-87- | 2,6-Octadien-1-ol, 3,7-dimethyl-, | Geranyl acetate | 0.0256 |
| | 3 | 1-acetate, (2E)- | | |
| 222 | 65443- | Cyclopentanone, 2,2,5-trimethyl- | Veloutone | 0.0261 |
| | 14-3 | 5-pentyl- | | |
| 223 | 144-39- | 1,6-Octadien-3-ol, 3,7-dimethyl-, | Linalyl Propionate | 0.0263 |
| | 8 | 3-propanoate | | |
| 224 | 61931- | 1,6-Nonadien-3-ol, 3,7-dimethyl-, | 3,7-Dimethyl-1,6- | 0.0263 |
| | 80-4 | 3-acetate | nonadien-3-yl | |
| | | | acetate | |
| 225 | 102-13- | Benzeneacetic acid, 2- | Iso Butyl | 0.0263 |
| | 6 | methylpropyl ester | Phenylacetate | |
| 226 | 104-55- | 2-Propenal, 3-phenyl- | Cinnamic | 0.0265 |
| | 2 | | Aldehyde | |
| 227 | 35044- | 1,3-Cyclohexadiene-1-carboxylic | Ethyl Safranate | 0.0266 |
| | 59-8 | acid, 2,6,6-trimethyl-, ethyl ester | | |
| 228 | 53767- | 7-Octen-2-ol, 2,6-dimethyl-, 2- | Dihydro Terpinyl | 0.0269 |
| | 93-4 | acetate | Acetate | |
| 229 | 39212- | 2(3H)-Furanone, 5-butyldihydro-4- | Methyl | 0.027 |
| | 23-2 | methyl- | Octalactone | |
| 230 | 122-97- | Benzenepropanol | Phenyl Propyl | 0.0271 |
| | 4 | | Alcohol | |
| 231 | 81786- | 3-Hepten-2-one, 3,4,5,6,6- | Koavone | 0.0275 |
| | 73-4 | pentamethyl-, (3Z)- | | |
| 232 | 98-55-5 | 3-Cyclohexene-1-methanol, α,α,4- | Alpha-Terpineol | 0.0283 |
| | | trimethyl- | | |
| 233 | 675-09- | 2H-Pyran-2-one, 4,6-dimethyl- | Levistamel | 0.0294 |
| | 2 | | | |
| 234 | 2463- | 2-Undecenal | 2-Undecene-1-al | 0.0297 |
| | 77-6 | | | |
| 235 | 120-72- | 1H-Indole | Indole | 0.0298 |
| | 9 | | | |
| 236 | 215231- | Cyclohexanol, 1-methyl-3-(2- | Rossitol | 0.0299 |
| | 33-7 | methylpropyl)- | | |
| 237 | 105-85- | 6-Octen-1-ol, 3,7-dimethyl-, 1- | Citronellyl Formate | 0.03 |
| | 1 | formate | | |
| 238 | 70214- | 2-Nonanol, 6,8-dimethyl- | Nonadyl | 0.0301 |
| | 77-6 | | | |
| 239 | 33885- | Bicyclo[3.1.1]hept-2-ene-2- | Pino Acetaldehyde | 0.0304 |
| | 51-7 | propanal, 6,6-dimethyl- | | |
| 240 | 67883- | 2-Butenoic acid, 2-methyl-, (3Z)- | Cis-3-Hexenyl | 0.0306 |
| | 79-8 | 3-hexen-1-yl ester, (2E)- | Tiglate | |
| 241 | 24717- | Propanoic acid, 2-methyl-, | Abierate | 0.0311 |
| | 86-0 | (1R,2S,4R)-1,7,7- | | |
| | | trimethylbicyclo[2.2.1]hept-2-yl | | |
| | | ester, rel- | | |
| 242 | 103-93- | Propanoic acid, 2-methyl-, 4- | Para Cresyl iso- | 0.0312 |
| | 5 | methylphenyl ester | Butyrate | |
| 243 | 3658- | 4-hydroxy-2,5-dimethylfuran-3- | Pineapple Ketone | 0.032 |
| | 77-3 | one | | |
| 244 | 2216- | Cyclohexanol, 5-methyl-2-(1- | L-Menthol | 0.0323 |
| | 51-5 | methylethyl)-, (1R,2S,5R)- | | |
| 245 | 1975- | Decanenitrile | Decanonitrile | 0.0325 |
| | 78-6 | | | |
| 246 | 141-25- | 3,7-Dimethyloct-6-en-1-ol | Rhodinol | 0.0329 |
| | 3 | | | |
| 247 | 53046- | 3,6-Nonadien-1-ol, (3Z,6Z)- | 3,6 Nonadien-1-ol | 0.0336 |
| | 97-2 | | | |
| 248 | 76649- | 3,6-Nonadien-1-ol | 3,6-Nonadien-1-ol | 0.0336 |
| | 25-7 | | | |
| 249 | 115-99- | 1,6-Octadien-3-ol, 3,7-dimethyl-, | Linalyl Formate | 0.0344 |
| | 1 | 3-formate | | |
| 250 | 112-54- | Dodecanal | Lauric Aldehyde | 0.0344 |
| | 9 | | | |
| 251 | 118-61- | Benzoic acid, 2-hydroxy-, ethyl | Ethyl salicylate | 0.0348 |
| | 6 | ester | | |
| 252 | 98-52-2 | Cyclohexanol, 4-(1,1- | Patchon | 0.0348 |
| | | dimethylethyl)- | | |
| 253 | 6378- | Hexyl hexanoate | Hexyl hexanoate | 0.0349 |
| | 65-0 | | | |
| 254 | 103-37- | Butanoic acid, phenylmethyl ester | Benzyl Butyrate | 0.0366 |
| | 7 | | | |
| 255 | 56011- | Benzene, [2-(3- | Phenyl Ethyl | 0.0369 |
| | 02-0 | methylbutoxy)ethyl]- | Isoamyl | |
| | | | Ether | |
| 256 | 82461- | Furan, tetrahydro-2,4-dimethyl-4- | Rhubafuran ® | 0.0378 |
| | 14-1 | phenyl- | | |
| 257 | 84560- | Cyclopentanol, 2-pentyl- | Cyclopentol | 0.0379 |
| | 00-9 | | | |
| 258 | 106-21- | 1-Octanol, 3,7-dimethyl- | Dimethyl Octanol | 0.0386 |
| | 8 | | | |
| 259 | 35044- | 2,4-Cyclohexadiene-1-carboxylic | Ethyl Safranate | 0.0388 |
| | 57-6 | acid, 2,6,6-trimethyl-, ethyl ester | | |
| 260 | 112-45- | 10-Undecenal | Undecylenic | 0.039 |
| | 8 | | aldehyde | |
| 261 | 80-26-2 | 3-Cyclohexene-1-methanol, α, α, | Terpinyl Acetate | 0.0392 |
| | | 4-trimethyl-, 1-acetate | | |
| 262 | 498-81- | Cyclohexanemethanol, α, α, 4- | Dihydro Terpineol | 0.0392 |
| | 7 | trimethyl- | | |
| 263 | 464-45- | Bicyclo[2.2.1]heptan-2-ol,1,7,7- | I-Borneol | 0.0398 |
| | 9 | trimethyl-, (1S,2R,4S)- | | |
| 264 | 124-76- | Bicyclo[2.2.1]heptan-2-ol, 1,7,7- | 1.7.7-Trimethyl- | 0.0398 |
| | 5 | trimethyl-, (1R,2R,4R)-rel- | Bicyclo-1.2.2- | |
| | | | Heptanol-2 | |
| 265 | 67874- | Cyclohexanol, 2-(1,1- | Coniferan | 0.0398 |
| | 72-0 | dimethylpropyl)-, 1-acetate | | |
| 266 | 67634- | Acetic acid, 2-(3-methylbutoxy)-, | Allyl Amyl | 0.04 |
| | 00-8 | 2-propen-1-yl ester | Glycolate | |
| 267 | 3613- | Octanal, 7-methoxy-3,7-dimethyl- | Methoxycitronellal | 0.0402 |
| | 30-7 | | | |
| 268 | 143-08- | 1-Nonanol | Nonyl Alcohol | 0.0407 |
| | 8 | | | |
| 269 | 104-50- | 2(3H)-Furanone, 5-butyldihydro- | Gamma | 0.0408 |
| | 7 | | Octalactone | |
| 270 | 22457- | 3-Heptanone, 5-methyl-, oxime | Stemone ® | 0.0414 |
| | 23-4 | | | |
| 271 | 41884- | 1-Hexanol, 5-methyl-2-(1- | Tetrahydro | 0.0423 |
| | 28-0 | methylethyl)-, (2R)- | Lavandulol | |
| 272 | 8000- | 2-(4-methyl-1-cyclohex-3- | Alpha Terpineol | 0.0432 |
| | 41-7 | enyl)propan-2-ol | | |
| 273 | 6784- | 3-Cyclohexene-1-propanal, Î²,4- | Limonenal | 0.0438 |
| | 13-0 | dimethyl- | | |
| 274 | 14374- | Benzene, 1-methyl-4-(1- | Verdoracine | 0.0446 |
| | 92-6 | methylethyl)-2-(1-propen-1-yl)- | | |
| 275 | 2756- | Bicyclo[2.2.1]heptan-2-ol,1,7,7- | Iso Bornyl | 0.0454 |
| | 56-1 | trimethyl-, 2-propanoate, | Propionate | |
| | | (1R,2R,4R)-rel- | | |
| 276 | 35852- | Pentanoic acid, (3Z)-3-hexen-1-yl | Cis-3-Hexenyl | 0.0458 |
| | 46-1 | ester | Valerate | |
| 277 | 68527- | 3-Cyclohexene-1-methanol, 2,4,6- | Isocyclogeraniol | 0.0464 |
| | 77-5 | trimethyl- | | |
| 278 | 562-74- | 3-Cyclohexen-1-ol, 4-methyl-1-(1- | Terpinenol-4 | 0.0478 |
| | 3 | methylethyl)- | | |
| 279 | 358331 | 2,5-Octadien-4-one, 5,6,7- | Pomarose | 0.0481 |
| | -95-0 | trimethyl-, (2E)- | | |
| 280 | 122-03- | Benzaldehyde, 4-(1-methylethyl)- | Cuminic Aldehyde | 0.0482 |
| | 2 | | | |
| 281 | 28462- | Bicyclo[2.2.1]heptan-2-ol, 1,2,3,3- | Humus Ether | 0.0487 |
| | 85-3 | tetramethyl-, (1R,2R,4S)-rel- | | |
| 282 | 104-62- | Formic acid, 2-phenylethyl ester | Phenyl Ethyl | 0.0505 |
| | 1 | | Formate | |
| 283 | 103-28- | Propanoic acid, 2-methyl-, | Benzyl Iso | 0.0513 |
| | 6 | phenylmethyl ester | Butyrate | |
| 284 | 7786- | 2,6-Nonadien-1-ol | 2,6-Nonadien-1-ol | 0.0537 |
| | 44-9 | | | |
| 285 | 13491- | Cyclohexanol, 2-(1,1- | Verdol | 0.0543 |
| | 79-7 | dimethylethyl)- | | |
| 286 | 2550- | 2-Butanone, 4-phenyl- | Benzyl Acetone | 0.0557 |
| | 26-7 | | | |
| 287 | 103-45- | Acetic acid, 2-phenylethyl ester | Phenyl Ethyl | 0.0564 |
| | 7 | | Acetate | |
| 288 | 111-80- | 2-Nonynoic acid, methylester | Methyl Octine | 0.0568 |
| | 8 | | Carbonate | |
| 289 | 16510- | Benzene, 1-(cyclopropylmethyl)- | Toscanol | 0.0587 |
| | 27-3 | 4-methoxy- | | |
| 290 | 208041 | Heptanenitrile, 2-propyl- | Jasmonitrile | 0.0592 |
| | -98-9 | | | |
| 291 | 53398- | Butanoic acid, 2-methyl-, (3Z)-3- | cis-3-Hexenyl | 0.0613 |
| | 85-9 | hexen-1-yl ester | Alpha | |
| | | | Methyl Butyrate | |
| 292 | 67019- | 2,6-Nonadienenitrile | Violet Nitrile | 0.062 |
| | 89-0 | | | |
| 293 | 13816- | Benzonitrile, 4-(1-methylethyl)- | Cumin Nitrile | 0.0623 |
| | 33-6 | | | |
| 294 | 3720- | 2-Cyclohexen-1-one, 3-methyl-5- | Livescone | 0.0627 |
| | 16-9 | propyl- | | |
| 295 | 62406- | 6, 10-Dioxaspiro[4.5]decane, 8,8- | Opalal CI | 0.0629 |
| | 73-9 | dimethyl-7-(1-methylethyl)- | | |
| 296 | 16587- | Cyclohexanone, 4-(1,1- | Orivone | 0.0649 |
| | 71-6 | dimethylpropyl)- | | |
| 297 | 6485- | 2-Cyclohexen-1-one, 2-methyl-5- | Laevo carvone | 0.0656 |
| | 40-1 | (1-methylethenyl)-, (5R)- | | |
| 298 | 15373- | 3-Cyclopentene-1-acetonitrile, | Cantryl ® | 0.067 |
| | 31-6 | 2,2,3-trimethyl- | | |
| 299 | 3913- | 2-Decenal, (2E)- | 2-Decene-1-al | 0.0674 |
| | 81-3 | | | |
| 300 | 7549- | 2,6-Octadiene, 1,1-dimethoxy-3,7- | Citral Dimethyl | 0.0678 |
| | 37-3 | dimethyl- | Acetal | |
| 301 | 25225- | Cyclohexanemethanol, α, 3,3- | Aphermate | 0.0678 |
| | 08-5 | trimethyl-, 1-formate | | |
| 302 | 119-36- | Benzoic acid, 2-hydroxy-, methyl | Methyl salicylate | 0.07 |
| | 8 | ester | | |
| 303 | 89-48-5 | Cyclohexanol, 5-methyl-2-(1- | Menthyl Acetate | 0.0707 |
| | | methylethyl)-, 1-acetate, | | |
| | | (1R,2S,5R)-rel- | | |
| 304 | 106-26- | 2,6-Octadienal, 3,7-dimethyl-, | Neral | 0.0712 |
| | 3 | (2Z)- | | |
| 305 | 5392- | 2,6-Octadienal, 3,7-dimethyl- | Citral | 0.0712 |
| | 40-5 | | | |
| 306 | 60-12-8 | 2-phenyl ethanol | Phenethyl alcohol | 0.0741 |
| | | | or | |
| | | | Phenylethyl | |
| | | | alcohol | |
| 307 | 22471- | Cyclohexanecarboxylic acid, | Thesaron | 0.0767 |
| | 55-2 | 2,2,6-trimethyl-, ethyl ester, | | |
| | | (1R,6S)-rel- | | |
| 308 | 80-57-9 | Bicyclo[3.1.1]hept-3-en-2-one, | Verbenone | 0.0773 |
| | | 4,6,6-trimethyl- | | |
| 309 | 5331- | Benzene, (2-butoxyethyl)- | Butyl phenethyl | 0.0776 |
| | 14-6 | | ether | |
| 310 | 35854- | 6-Nonen-1-ol, (6Z)- | cis-6-None-1-ol | 0.0777 |
| | 86-5 | | | |
| 311 | 98-53-3 | Cyclohexanone, 4-(1,1- | Patchi | 0.0778 |
| | | dimethylethyl)- | | |
| 312 | 32669- | Ethanone, 1-(3-cycloocten-1-yl)- | Tanaisone ® | 0.0815 |
| | 00-4 | | | |
| 313 | 112-44- | Undecanal | Undecyl Aldehyde | 0.0832 |
| | 7 | | | |
| 314 | 67634- | 3-Cyclohexene-1-methanol, 3,5- | Floralate | 0.085 |
| | 25-7 | dimethyl-, 1-acetate | | |
| 315 | 188570- | Cyclopropanecarboxylic acid, | Montaverdi | 0.0864 |
| | 78-7 | (3Z)-3-hexen-1-yl ester | | |
| 316 | 100-86- | Benzeneethanol, α,α-dimethyl- | Dimethyl Benzyl | 0.0888 |
| | 7 | | Carbinol | |
| 317 | 101-97- | Benzeneacetic acid, ethyl | Ethyl Phenyl | 0.0897 |
| | 3 | ester | Acetate | |
| 318 | 78-70-6 | 1,6-Octadien-3-ol, 3,7-dimethyl- | Linalool | 0.0905 |
| 319 | 125-12- | Bicyclo[2.2.1]heptan-2-ol, 1,7,7- | Iso Bornyl Acetate | 0.0959 |
| | 2 | trimethyl-, 2-acetate, (1R,2R,4R)- | | |
| | | rel- | | |
| 320 | 103-05- | Benzenepropanol, α,α-dimethyl- | Phenyl Ethyl | 0.0977 |
| | 9 | | Dimethyl | |
| | | | Carbinol | |
| 321 | 112-12- | 2-Undecanone | Methyl Nonyl | 0.0978 |
| | 9 | | Ketone | |
| 322 | 24168- | Pyrazine, 2-methoxy-3-(1- | Methoxyisobutylpy | 0.0995 |
| | 70-5 | methylpropyl)- | razine | |
| 323 | 112-44- | Undecanal | n-Undecanal | 0.102 |
| | 7 | | | |
| 324 | 80-25-1 | Cyclohexanemethanol, α,α,4- | Menthanyl Acetate | 0.103 |
| | | trimethyl-, 1-acetate | | |
| 325 | 88-41-5 | Cyclohexanol, 2-(1,1- | Verdox ™ | 0.103 |
| | | dimethylethyl)-, 1-acetate | | |
| 326 | 32210- | Cyclohexanol, 4-(1,1- | Vertenex | 0.103 |
| | 23-4 | dimethylethyl)-, 1-acetate | | |
| 327 | 71159- | 3-Cyclohexene-1-methanethiol, | Grapefruit | 0.105 |
| | 90-5 | α,α,4-trimethyl- | mercaptan | |
| 328 | 111-87- | 1-Octanol | Octyl Alcohol | 0.114 |
| | 5 | | | |
| 329 | 18479- | 2-Octanol, 2,6-dimethyl- | Tetra-Hydro | 0.115 |
| | 57-7 | | Myrcenol | |
| 330 | 78-69-3 | 3,7-dimethyloctan-3-ol | Tetra-Hydro | 0.115 |
| | | | Linalool | |
| 331 | 115-95- | 1,6-Octadien-3-ol, 3,7-dimethyl-, | Linalyl acetate | 0.116 |
| | 7 | 3-acetate | | |
| 332 | 13851- | Bicyclo[2.2.1]heptan-2-ol, 1,3,3- | Fenchyl Acetate | 0.117 |
| | 11-1 | trimethyl-, 2-acetate | | |
| 333 | 62439- | Heptanal, 6-methoxy-2,6- | Methoxy Melonal | 0.119 |
| | 41-2 | dimethyl- | | |
| 334 | 59323- | 1,3-Oxathiane, 2-methyl-4-propyl-, | Oxane | 0.123 |
| | 76-1 | (2R,4S)-rel- | | |
| 335 | 111-12- | 2-Octynoic acid, methyl ester | Methyl Heptine | 0.125 |
| | 6 | | Carbonate | |
| 336 | 16491- | Butanoic acid, (3Z)-3-hexen-1-yl | Cis 3 Hexenyl | 0.135 |
| | 36-4 | ester | Butyrate | |
| 337 | 111-79- | 2-Nonenoic acid, methyl ester | Methyl-2- | 0.146 |
| | 5 | | Nonenoate | |
| 338 | 695-06- | 2(3H)-Furanone, 5-ethyldihydro- | Gamma | 0.152 |
| | 7 | | Hexalactone | |
| 339 | 21722- | Cyclohexaneethanol, 1-acetate | Cyclohexyl Ethyl | 0.152 |
| | 83-8 | | Acetate | |
| 340 | 100-51- | Benzenemethanol | Benzyl Alcohol | 0.158 |
| | 6 | | | |
| 341 | 10032- | Butanoic acid, 2-methyl-, hexyl | Hexyl-2-Methyl | 0.158 |
| | 15-2 | ester | Butyrate | |
| 342 | 20125- | 3-Octen-1-ol, (3Z)- | Octenol | 0.16 |
| | 84-2 | | | |
| 343 | 142-19- | Heptanoic acid, 2-propen-1-yl | Allyl Heptoate | 0.16 |
| | 8 | ester | | |
| 344 | 14765- | Cyclohexanone, 2-(1- | 2-sec-Butyl Cyclo | 0.163 |
| | 30-1 | methylpropyl)- | Hexanone | |
| 345 | 140-11- | Acetic acid, phenylmethyl ester | Benzyl acetate | 0.164 |
| | 4 | | | |
| 346 | 18479- | 7-Octen-2-ol, 2,6-dimethyl- | Dihydromyrcenol | 0.166 |
| | 58-8 | | | |
| 347 | 35158- | 2-Hexenal, 5-methyl-2-(1- | Lactone of Cis | 0.172 |
| | 25-9 | methylethyl)- | Jasmone | |
| 348 | 40853- | 1-Hexanol, 5-methyl-2-(1- | Tetrahydro | 0.173 |
| | 55-2 | methylethyl)-, 1-acetate | Lavandulyl | |
| | | | Acetate | |
| 349 | 933-48- | Cyclohexanol, 3,3,5-trimethyl-, | Trimethylcyclohex | 0.173 |
| | 2 | (1R,5R)-rel- | anol | |
| 350 | 101-41- | Methyl 2-phenylacetate | Methyl 2- | 0.176 |
| | 7 | | phenylacetate | |
| 351 | 93-89-0 | Benzoic acid, ethyl ester | Ethyl Benzoate | 0.18 |
| 352 | 20780- | 3-Octanol, 3,7-dimethyl-, 3- | Tetrahydro Linalyl | 0.18 |
| | 48-7 | acetate | Acetate | |
| 353 | 41519- | Propanoic acid, 2-methyl-, (3Z)-3- | Hexenyl | 0.182 |
| | 23-7 | hexen-1-yl ester | Isobutyrate | |
| 354 | 24237- | 2H-Pyran, 6-butyl-3,6-dihydro-2,4- | Gyrane | 0.186 |
| | 00-1 | dimethyl- | | |
| 355 | 122-00- | Ethanone, 1-(4-methylphenyl)- | Para Methyl | 0.187 |
| | 9 | | Acetophenone | |
| 356 | 143-13- | Acetic acid, nonyl ester | Nonyl Acetate | 0.197 |
| | 5 | | | |
| 357 | 93-92-5 | Benzenemethanol, α-methyl-, 1- | Methylphenylcarbinol | 0.203 |
| | | acetate | Acetate | |
| 358 | 16251- | Benzenepropanal, β-methyl- | Trifernal | 0.206 |
| | 77-7 | | | |
| 359 | 112-31- | Decanal | Decyl Aldehyde | 0.207 |
| | 2 | | | |
| 360 | 67674- | 2-Hexene, 6,6-dimethoxy-2,5,5- | Methyl | 0.214 |
| | 46-8 | trimethyl- | Pamplemousse | |
| 361 | 76-22-2 | Bicyclo[2.2.1]heptan-2-one, 1,7,7- | Camphor gum | 0.225 |
| | | trimethyl- | | |
| 362 | 64988- | Benzene, 1-(ethoxymethyl)-2- | Rosacene | 0.246 |
| | 06-3 | methoxy- | | |
| 363 | 55739- | Cyclohexanone, 2-ethyl-4,4- | Thuyacetone | 0.25 |
| | 89-4 | dimethyl- | | |
| 364 | 150-78- | Benzene, 1,4-dimethoxy- | Hydroquinone | 0.25 |
| | 7 | | Dimethyl | |
| | | | Ether | |
| 365 | 89-80-5 | Cyclohexanone, 5-methyl-2-(1- | Menthone | 0.256 |
| | | methylethyl)-, (2R,5S)-rel- | Racemic | |
| 366 | 2463- | 2-Nonenal | 2 Nonen-1-al | 0.256 |
| | 53-8 | | | |
| 367 | 104-45- | Benzene, 1-methoxy-4-propyl- | Dihydroanethole | 0.266 |
| | 0 | | | |
| 368 | 24683- | Pyrazine, 2-methoxy-3-(2- | 2-Methoxy-3- | 0.273 |
| | 00-9 | methylpropyl)- | Isobutyl | |
| | | | Pyrazine | |
| 369 | 104-57- | Formic acid, phenylmethyl ester | Benzyl Formate | 0.273 |
| | 4 | | | |
| 370 | 557-48- | 2,6-Nonadienal, (2E,6Z)- | E Z-2,6-Nonadien- | 0.28 |
| | 2 | | 1-al | |
| 371 | 80118- | Propanoic acid, 2-methyl-, 1,3- | Iso Pentyrate | 0.285 |
| | 06-5 | dimethyl-3-buten-1-yl ester | | |
| 372 | 93-53-8 | Benzeneacetaldehyde, α-methyl- | Hydratropic | 0.294 |
| | | | aldehyde | |
| 373 | 98-86-2 | Ethanone, 1-phenyl- | Acetophenone | 0.299 |
| 374 | 54546- | 1,3-Dioxane, 2-butyl-4,4,6- | Herboxane | 0.33 |
| | 26-8 | trimethyl- | | |
| 375 | 13254- | 2-Heptanol, 2,6-dimethyl- | Dimethyl-2,6- | 0.33 |
| | 34-7 | | Heptan-2-ol | |
| 376 | 65405- | 4-Decenal, (4E)- | Decenal (Trans-4) | 0.331 |
| | 70-1 | | | |
| 377 | 16356- | 1,3,5-Undecatriene | Undecatriene | 0.336 |
| | 11-9 | | | |
| 378 | 93-58-3 | Benzoic acid, methyl ester | Methyl Benzoate | 0.34 |
| 379 | 105-53- | Propanedioic acid, 1,3-diethyl | Diethyl Malonate | 0.344 |
| | 3 | ester | | |
| 380 | 5405- | Butanoic acid, 3-hydroxy-, ethyl | Ethyl-3-Hydroxy | 0.362 |
| | 41-4 | ester | Butyrate | |
| 381 | 122-78- | Benzeneacetaldehyde | Phenyl | 0.368 |
| | 1 | | acetaldehyde | |
| 382 | 23250- | Cyclohexanecarboxylic acid, 1,4- | Cyprissate | 0.405 |
| | 42-2 | dimethyl-, methyl ester, trans- | | |
| 383 | 105-79- | Hexanoic acid, 2-methylpropyl | Iso Butyl Caproate | 0.413 |
| | 3 | ester | | |
| 384 | 2349- | Propanoic acid, 2-methyl-, hexyl | Hexyl isobutyrate | 0.413 |
| | 07-7 | ester | | |
| 385 | 10250- | 4-Heptanol, 2,6-dimethyl-, 4- | Alicate | 0.454 |
| | 45-0 | acetate | | |
| 386 | 58430- | 1-Hexanol, 3,5,5-trimethyl-, 1- | Iso Nonyl Acetate | 0.47 |
| | 94-7 | acetate | | |
| 387 | 589-98- | 3-Octanol | Octanol-3 | 0.512 |
| | 0 | | | |
| 388 | 100-47- | Benzonitrile | Benzyl Nitrile | 0.524 |
| | 0 | | | |
| 389 | 16409- | 2H-Pyran, tetrahydro-4-methyl-2- | Rose Oxide | 0.551 |
| | 43-1 | (2-methyl-1-propen-1-yl)- | | |
| 390 | 101-48- | Benzene, (2,2-dimethoxyethyl)- | Phenyl | 0.556 |
| | 4 | | Acetaldehyde | |
| | | | Dimethyl Acetal | |
| 391 | 68039- | 3-Cyclohexene-1- | Ligustral or Triplal | 0.578 |
| | 49-6 | carboxaldehyde, 2,4-dimethyl- | | |
| 392 | 106-30- | Heptanoic acid, ethyl ester | Ethyl Oenanthate | 0.602 |
| | 9 | | | |
| 393 | 106-72- | 5-Heptenal, 2,6-dimethyl- | Melonal | 0.622 |
| | 9 | | | |
| 394 | 123-68- | Hexanoic acid, 2-propen-1-yl | Allyl Caproate | 0.678 |
| | 2 | ester | | |
| 395 | 67633- | Carbonic acid, (3Z)-3-hexen-1-yl | Liffarome ™ | 0.721 |
| | 96-9 | methyl ester | | |
| 396 | 189440- | 4,7-Octadienoic acid, methyl | Anapear | 0.777 |
| | 77- | ester, (4E)- | | |
| | 5 | | | |
| 397 | 69103- | Oxirane, 2,2-dimethyl-3-(3- | Myroxide | 0.806 |
| | 20-4 | methyl-2,4-pentadien-1-yl)- | | |
| 398 | 3016- | 2,4,6-Octatriene, 2,6-dimethyl-, | Allo-Ocimene | 0.816 |
| | 19-1 | (4E,6E)- | | |
| 399 | 19872- | 2-Pentanone, 4-mercapto-4- | 4-Methyl-4- | 0.843 |
| | 52-7 | methyl- | Mercaptopentan-2- | |
| | | | one | |
| | | | 1ppm TEC | |
| 400 | 24691- | Cyclohexane, 3-ethoxy-1,1,5- | Herbavert | 0.852 |
| | 15-4 | trimethyl-, cis- (9CI) | | |
| 401 | 928-95- | 2-Hexen-1-ol, (2E)- | Trans-2-Hexenol | 0.873 |
| | 0 | | | |
| 402 | 928-94- | 2-Hexen-1-ol, (2Z)- | Beta Gamma | 0.873 |
| | 9 | | Hexenol | |
| 403 | 141-97- | Butanoic acid, 3-oxo-, ethyl ester | Ethyl Acetoacetate | 0.89 |
| | 9 | | | |
| 404 | 100-52- | Benzaldehyde | Benzaldehyde | 0.974 |
| | 7 | | | |
| 405 | 928-96- | 3-Hexen-1-ol, (3Z)- | cis-3-Hexenol | 1.04 |
| | 1 | | | |
| 406 | 18640- | Thiazole, 2-(2-methylpropyl)- | 2-Isobutylthiazole | 1.07 |
| | 74-9 | | | |
| 407 | 99-85-4 | 1,4-Cyclohexadiene, 1-methyl-4- | Gamma Terpinene | 1.08 |
| | | (1-methylethyl)- | | |
| 408 | 51115- | Butanoic acid, 2-methylbutyl ester | Amyl butyrate | 1.09 |
| | 64-1 | | | |
| 409 | 106-27- | Butanoic acid, 3-methylbutyl ester | Amyl Butyrate | 1.09 |
| | 4 | | | |
| 410 | 97-64-3 | Propanoic acid, 2-hydroxy-, ethyl | Ethyl Lactate | 1.16 |
| | | ester | | |
| 411 | 3681- | 3-Hexen-1-ol, 1-acetate, (3Z)- | cis-3-Hexenyl | 1.22 |
| | 71-8 | | acetate | |
| 412 | 81925- | 2-Hepten-4-one, 5-methyl- | Filbertone 1% in | 1.25 |
| | 81-7 | | TEC | |
| 413 | 110-93- | 5-Hepten-2-one, 6-methyl- | Methyl Heptenone | 1.28 |
| | 0 | | | |
| 414 | 142-92- | Acetic acid, hexyl ester | Hexyl acetate | 1.39 |
| | 7 | | | |
| 415 | 110-41- | Undecanal, 2-methyl- | Methyl Nonyl | 1.43 |
| | 8 | | Acetaldehyde | |
| 416 | 106-68- | 3-Octanone | Ethyl Amyl Ketone | 1.5 |
| | 3 | | | |
| 417 | 138-86- | Cyclohexene, 1-methyl-4-(1- | dl-Limonene | 1.54 |
| | 3 | methylethenyl)- | | |
| 418 | 5989- | Cyclohexene, 1-methyl-4-(1- | d-limonene | 1.54 |
| | 27-5 | methylethenyl)-, (4R)- | | |
| 419 | 13877- | 1,3,6-Octatriene, 3,7-dimethyl- | Ocimene | 1.56 |
| | 91-3 | | | |
| 420 | 470-82- | 2-Oxabicyclo[2.2.2]octane, 1,3,3- | Eucalyptol | 1.65 |
| | 6 | trimethyl- | | |
| 421 | 99-87-6 | Benzene, 1-methyl-4-(1- | Para Cymene | 1.65 |
| | | methylethyl)- | | |
| 422 | 104-93- | Benzene, 1-methoxy-4-methyl- | Para Cresyl Methyl | 1.65 |
| | 8 | | Ether | |
| 423 | 123-66- | Hexanoic acid, ethyl ester | Ethyl Caproate | 1.66 |
| | 0 | | | |
| 424 | 111-13- | 2-Octanone | Methyl Hexyl | 1.72 |
| | 7 | | Ketone | |
| 425 | 7392- | 2H-Pyran, 2-ethenyltetrahydro- | Limetol | 1.9 |
| | 19-0 | 2,6,6-trimethyl- | | |
| 426 | 124-13- | Octanal | Octyl Aldehyde | 2.07 |
| | 0 | | | |
| 427 | 123-35- | 1,6-Octadiene, 7-methyl-3- | Myrcene | 2.29 |
| | 3 | methylene- | | |
| 428 | 105-68- | 1-Butanol, 3-methyl-, 1- | Amyl Propionate | 2.36 |
| | 0 | propanoate | | |
| 429 | 127-91- | Bicyclo[3.1.1]heptane, 6,6- | Beta Pinene | 2.4 |
| | 3 | dimethyl-2-methylene- | | |
| 430 | 3387- | Bicyclo[3.1.0]hexane, 4- | Sabinene | 2.63 |
| | 41-5 | methylene-1-(1-methylethyl)- | | |
| 431 | 39255- | Pentanoic acid, 2-methyl-, ethyl | Manzanate | 2.91 |
| | 32-8 | ester | | |
| 432 | 79-92-5 | Bicyclo[2.2.1]heptane, 2,2- | Camphene | 3.38 |
| | | dimethyl-3-methylene- | | |
| 433 | 7785- | Bicyclo[3.1.1]hept-2-ene, 2,6,6- | Alpha Pinene | 3.49 |
| | 70-8 | trimethyl-, (1R,5R)- | | |
| 434 | 57366- | 1,3-Dioxolane-2-methanamine, N- | Methyl Dioxolan | 3.88 |
| | 77-5 | methyl- | | |
| 435 | 1191- | 2-Buten-1-ol, 3-methyl-, 1-acetate | Prenyl acetate | 3.99 |
| | 16-8 | | | |
| 436 | 123-51- | 1-Butanol, 3-methyl- | Isoamyl Alcohol | 4.16 |
| | 3 | | | |
| 437 | 6728- | 2-Hexenal, (2E)- | Trans-2 Hexenal | 4.62 |
| | 26-3 | | | |
| 438 | 110-43- | 2-Heptanone | Methyl Amyl | 4.73 |
| | 0 | | Ketone | |
| 439 | 66576- | Butanoic acid, 2-methyl-, 1- | Iso Propyl 2- | 5.1 |
| | 71-4 | methylethyl ester | Methylbutyrate | |
| 440 | 123-92- | 1-Butanol, 3-methyl-, 1-acetate | Iso Amyl Acetate | 5.68 |
| | 2 | | | |
| 441 | 7452- | Butanoic acid, 2-methyl-, ethyl | Ethyl-2-Methyl | 7.85 |
| | 79-1 | ester | Butyrate | |
| 442 | 14765- | 1-Butanol | Butyl Alcohol | 8.52 |
| | 30-1 | | | |
| 443 | 105-54- | Butanoic acid, ethyl ester | Ethyl Butyrate | 13.9 |
| | 4 | | | |
| 444 | 110-19- | Acetic acid, 2- | Isobutyl Acetate | 18 |
| | 0 | methylpropyl ester | | |
| 445 | 105-37- | Propanoic acid, ethyl ester | Ethyl Propionate | 44.5 |
| | 3 | | | |
| 446 | 141-78- | Acetic acid ethyl ester | Ethyl Acetate | 112 |
| | 6 | | | |
| 447 | 75-18-3 | Methane, 1,1′-thiobis- | Dimethyl Sulfide | 647 |
| | | | 1.0% In DEP | |
| 448 | 107-31- | Formic acid, methyl ester | Methyl Formate | 732 |
| | 3 | | | |

**Origin: Perfumery ingredients may be obtained from one or more of the following companies: Firmenich (Geneva, Switzerland), Symrise AG (Holzminden, Germany), Givaudan (Argenteuil, France), IFF (Hazlet, New Jersey), Bedoukian (Danbury, Connecticut), Sigma Aldrich (St. Louis, Missouri), Millennium Speciality Chemicals (Olympia Fields, Illinois), Polarone International (Jersey City, New Jersey), and Aroma & Flavor Specialities (Danbury, Connecticut).

In an embodiment, the fragrance component comprises one or more perfumery ingredient selected from 6-Octen-1-ol, 3,7-dimethyl-; Benzoic acid, 2-(methylamino)-, methylester; 1,6-Octadien-3-ol, 3,7-dimethyl-; 1,6-Octadien-3-ol, 3,7-dimethyl-, 3-acetate; Acetic acid, phenylmethyl ester; Decanal; Cyclohexene, 1-methyl-4-(1-methylethenyl)-; Cyclohexene, 1-methyl-4-(1-methylethenyl)-, (4R)-; Octanal; and mixture thereof.

The amount of the fragrance component in the fragrance composition is not particularly limited. However, in an embodiment, the fragrance component is present in an amount of less than 50%, typically from 1% to 50%, more typically from 1% to 15%, by weight relative to the total weight of the fragrance composition.

The volatile solvent with a vapor pressure above 0.1 mmHg at 20 °C is present in an amount not greater than 70%. In an embodiment, the volatile solvent with a vapor pressure above 0.1 mmHg at 20 °C is present in an amount of from 20% to 70%, by weight relative to the total weight of the fragrance composition. In another embodiment, the volatile solvent with a vapor pressure above 0.1 mmHg at 20 °C is present in an amount of from 50% to 70%, by weight relative to the total weight of the fragrance composition. In another embodiment, the volatile solvent with a vapor pressure above 0.1 mmHg at 20 °C is in an amount not greater than 50%. In an embodiment, the volatile solvent with a vapor pressure above 0.1 mmHg at 20 °C is present in an amount of from 20% to 50%, by weight relative to the total weight of the fragrance composition. In an embodiment, the volatile solvent with a vapor pressure above 0.1 mmHg at 20 °C is present in an amount of from 40%, 41%, 42%, 43%, 44%, 45%, 46%, 47%, 48%, or 49%, to 50%, by weight relative to the total weight of the fragrance composition.

Exemplary volatile solvents with a vapor pressure above 0.1 mmHg at 20 °C include, but are not limited to, ethanol, methanol, propanol, isopropanol, butanol, and mixtures thereof. In an embodiment, the volatile solvent with a vapor pressure above 0.1 mmHg at 20 °C comprises ethanol.

In an embodiment, ethanol is present in an amount of from 40%, 41%, 42%, 43%, 44%, 45%, 46%, 47%, 48%, or 49%, to 50%, by weight relative to the total weight of the fragrance composition.

The fragrance composition according to the present disclosure may further comprise at least one amphiphilic organic solvent different from 1,3-butanediol and that is characterized by a log P greater than or equal to −2 and less than or equal to 2.

As used herein, “log P” refers to the logarithm (base 10) of the partition coefficient (P), which is defined as the ratio of a compound's organic (typically, oil)-to-aqueous phase concentrations. Log P describes the partition of a compound in a two-phase system made of octanol and water (Log P=Log (C_{oct}/C_{water})). This parameter provides a measure of the hydrophilic/lipophilic character of a compound: the higher the Log P, the more lipophilic the compound. Log P values may be determined empirically or calculated. In some embodiments, the log P values are calculated. Calculated log P, or C log P, may be obtained for each single perfuming ingredient according to methods known to those of ordinary skill in the art. For example, C log P can be obtained according to the program EPI suite (4.0); EPA (US Environmental Protection Agency) and Syracuse Research Corporation (SRC), 2000. In another example, C log P may be calculated according to a method described by Suzuki T. 1992, CHEMICALC 2, QCPE Program No 608, Department of chemistry, Indiana University; Suzuki T., Kudo Y. J. Comput.-Aided Mol. Design 1990, 4, 155; Suzuki T., J. Comput.-Aided Mol. Design 1991, 5, 149. In yet another example, C log P may be determined using an application available at the following website: http://www.daylight.com/daycgi/clogp.

In an embodiment, the at least one amphiphilic organic solvent is selected from the group consisting of glycols, typically 1,2-alkanediols, such as 1,2-propanediol, 1,2-butanediol, 1,2-pentanediol, 1,2-hexanediol, 1,2-heptanediol, and 1,2-octanediol, and 1,3-alkanediols, such as 1,3-propanediol, 2-methyl-1,3-propanediol, and 3-methyl-1,3-butanediol; 1,4-alkanediols, such as 1,4-butanediol; dipropylene glycol, polyalkylene glycols, typically polyethylene glycols, polypropylene glycols, and poly(ethylene/propylene) glycols; C₁-C₁₀ alkyl esters of citric acid, typically triethyl citrate, and combinations thereof.

In an embodiment, the fragrance composition further comprises a 1,2-alkanediol, typically 1,2-propanediol. In some embodiments, the fragrance composition further comprises 1,2-propanediol and one or more amphiphilic organic solvents selected from the group consisting of glycols, typically 1,2-alkanediols, such as 1,2-butanediol, 1,2-pentanediol, 1,2-hexanediol, 1,2-heptanediol, and 1,2-octanediol, and 1,3-alkanediols, such as 1,3-propanediol, 2-methyl-1,3-propanediol, and 3-methyl-1,3-butanediol; 1,4-alkanediols, such as 1,4-butanediol; dipropylene glycol, polyalkylene glycols, typically polyethylene glycols, polypropylene glycols, and poly(ethylene/propylene) glycols; C₁-C₁₀ alkyl esters of citric acid, typically triethyl citrate, and combinations thereof.

The amount of the at least one amphiphilic organic solvent is not particularly limited. However, in an embodiment, the at least one amphiphilic organic solvent is present in an amount of from 0.1% to 50%, typically 0.1% to 30%, more typically 5% to 15%, by weight relative to the total weight of the fragrance composition.

The fragrance composition may further comprise a perfuming co-ingredient. As used herein, a perfuming co-ingredient refers to ingredients that impart a hedonic effect, i.e., used for the primary purpose of conferring or modulating an odor. In other words, such a co-ingredient, to be considered as being a perfuming one, must be recognized by a person skilled in the art as being able to impart or modify in a positive or pleasant way the odor of a composition, and not just as having an odor. Perfuming co-ingredients may impart an additional benefit beyond that of modifying or imparting an odor, such as long-lasting, blooming, malodour counteraction, antimicrobial effect, antiviral effect, microbial stability, or pest control.

The nature and type of the perfuming co-ingredients do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds, and essential oils. Perfuming co-ingredients can be of natural or synthetic origin. Suitable perfumery co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release, in a controlled manner, various types of perfuming compounds, known as properfume or profragrance.

In an embodiment, the fragrance composition further comprises at least one fragrance modulator.

Fragrance modulators, also known as fixatives, are agents having the capacity to affect the manner in which the odor, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived.

Examples of fragrance modulators suitable for use according to the present disclosure include, but are not limited to, caprylyl alcohol, octanol, butyloctanol, isotridecyl alcohol, hexyldecanol, isostearyl alcohol, octyldecanol, octyldodecanol, decyltetradecanol, tetradecyloctadecanol, PPG-20 methyl glucose ether, methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and any combination thereof.

In an embodiment, the at least one fragrance modulator is hexyldecanol.

The fragrance composition may further comprise water. In an embodiment, water is present in an amount of from 0.1% to 99%, typically 0.1% to 30%, more typically 10% to 30%, by weight relative to the total weight of the fragrance composition.

The fragrance composition described herein may comprise a solid carrier. The fragrance composition or some element of the fragrance composition, such as the fragrance component, can be chemically or physically bound. In general, such solid carriers are employed either to stabilize the composition, or to control the rate of evaporation of the compositions or of some ingredients. Solid carriers are of current use in the art and a person skilled in the art knows how to reach the desired effect. Suitable solid carriers include, but are not limited to, absorbing gums or polymers or inorganic materials, such as porous polymers, cyclodextrines, dextrines, maltodextrines wood-based materials, organic or inorganic gels, clays, gypsum talc or zeolites.

Other suitable solid carriers include encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulphate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, carbohydrates, saccharides such as sucrose, mono-, di-, and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol, and isomalt, polyethylene glycol (PEG), polyvinyl pyrrolidin (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans, citric acid or any water soluble solid acid, fatty alcohols or fatty acids and mixtures thereof.

Other suitable encapsulating materials are described in reference texts known to those of skill in the art, such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag Gmbh & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

Other exemplary solid carriers include core-shell capsules with resins of aminoplast, polyamide, polyester, polyurea or polyurethane type, and mixtures thereof, made using techniques well-known to those of ordinary skill in the art, such as phase separation induced by polymerization, interfacial polymerization, coacervation, or a combination thereof, optionally in the presence of a polymeric stabilizer or of a cationic copolymer.

Resins may be produced by the polycondensation of an aldehyde (e.g., formaldehyde, 2,2-dimethoxyethanol, glyoxal, glyoxylic acid or glycolaldehyde, and mixtures thereof) with an amine such as urea, benzoguanamine, glycoluryl, melamine, methylol melamine, methylated methylol melamine, guanazole and the like, as well as mixtures thereof. Alternatively, one may use preformed resins like alkylolated polyamines such as those commercially available under the trademark Urac® (origin: Cytec Technology Corp.), Cymel® (origin: Cytec Technology Corp.), Urecoll® or Luracoll® (origin: BASF).

Other suitable resins are the those produced by the polycondensation of a polyol, like glycerol, and a polyisocyanate, for example, a trimer of hexamethylene diisocyanate, a trimer of isophorone diisocyanate or xylylene diisocyanate or a Biuret of hexamethylene diisocyanate or a trimer of xylylene diisocyanate with trimethylolpropane (marketed as Takenate® by Mitsui Chemicals), among which a trimer of xylylene diisocyanate with trimethylolpropane and a Biuret of hexamethylene diisocyanate are of mention.

The encapsulation of perfumes by polycondensation of amino resins, namely melamine-based resins with aldehydes is well-known in the art. Pertinent publications include, but are not limited to, K. Dietrich et al. Acta Polymerica, 1989, vol. 40, pages 243, 325 and 683, as well as 1990, vol. 41, page 91 and U.S. Pat. No. 4,396,670 issued Aug. 2, 1983. The general knowledge in encapsulation technology is very significant and cannot be exhaustive. More recent publications of pertinence, which disclose suitable uses of such microcapsules, are represented, for example, by the article of K. Bruyninckx and M. Dusselier, ACS Sustainable Chemistry & Engineering, 2019, vol. 7, pages 8041-8054. These publications are incorporated herein by reference.

The fragrance composition may optionally comprise at least one perfumery adjuvant.

The at least one perfumery adjuvant is an ingredient capable of imparting an additional added benefit such as a color, a particular light resistance, chemical stability, etc. A detailed description of the nature and type of adjuvant commonly used in perfuming compositions cannot be exhaustive, but it has to be mentioned that said ingredients are well known to a person skilled in the art.

Exemplary perfumery adjuvants include, but are not limited to, viscosity agents (e.g., surfactants, thickeners, gelling and/or rheology modifiers), stabilizing agents (e.g., preservatives, antioxidant, heat/light and or buffers or chelating agents, such as BHT), coloring agents (e.g., dyes and/or pigments), preservatives (e.g. antibacterial or antimicrobial or antifungal or anti irritant agents), abrasives, skin cooling agents, insect repellants, ointments, vitamins and mixtures thereof.

The fragrance compositions according to the present disclosure may be prepared according to any method known to those of ordinary skill in the art. The ordinarily-skilled artisan is perfectly able to design optimal formulations for the desired effect by admixing the above mentioned components to arrive at the desired composition by applying standard knowledge and concepts known to those of ordinary skill and by utilizing routine optimization methodologies.

In the second aspect, the present disclosure relates to a consumer product comprising the fragrance composition described herein.

The form of the consumer product is not particularly limited. In an embodiment, the consumer product is a perfume, a body-care product, a cosmetic preparation, a skin-care product, a fabric care product, an air care product, or a home care product.

In another embodiment, the consumer product is a fine perfume, a splash, an eau de toilette, an eau de parfum, a cologne, a body mist, a body spray, a hair mist, a shave or after-shave lotion, a shampoo, a coloring preparation, a color care product, a hair shaping product, a dental care product, a disinfectant, an intimate care product, a hair spray, a vanishing cream, a deodorant or antiperspirant, hair remover, tanning or sun product, a nail product, a skin cleansing, a makeup, a perfumed soap, a shower or bath mousse, oil or gel, a foot/hand care products, a hygiene product, a liquid or solid or unit-dose detergent, a fabric softener, a solid or liquid fabric scent-booster, a fabric refresher, an ironing water, an air freshener, a “ready to use” powdered air freshener, a mold remover, a furniture care product, a wipe, a dish detergent or hard-surface detergent, a leather care product, or a car care product.

The amount of the fragrance composition in the consumer product is not particularly limited. In an embodiment, the consumer product comprises the fragrance composition in an amount from 1% to 100%, typically 1% to 95%, more typically from 2% to 80%, still more typically from 3% to 70%, by weight relative to the total weight of the consumer product.

In an embodiment, the consumer product comprises the fragrance composition in an amount from 1% to 30%, typically from 2% to 20%, more typically from 3% to 10%, by weight relative to the total weight of the consumer product.

In the third aspect, the present disclosure relates to a method for enhancing or modulating the perceived olfactive impact and/or long-lastingness of a fragrance composition, the method comprising combining 1,3-butanediol with a fragrance component, and a volatile solvent with a vapor pressure above 0.1 mmHg at 20 °C to obtain the fragrance composition, wherein the volatile solvent with a vapor pressure above 0.1 mmHg at 20 °C is in an amount not greater than 70%, typically 20% to 70%, by weight relative to the total weight of the fragrance composition.

Generally, combining 1,3-butanediol with the fragrance component and the volatile solvent with a vapor pressure above 0.1 mmHg at 20 °C may be achieve using any suitable method known to those of ordinary skill in the art. For example, the components may be weighed and then mixed, typically by stirring, until homogeneous.

The fragrance compositions according to the present disclosure are generally homogeneous and transparent. As used herein, the term “homogeneous” means that all components of the composition are completely solubilized and the composition is uniform throughout. As used herein, the term “transparent” means that the composition has the property of transmitting light without appreciable light scattering. Transparency of a composition can be assessed by determining its turbidity expressed in NTU (Nephelometric Turbidity Units) using a turbidimeter, measured in a 2.5 cm cell at 25 °C, at wavelengths between 400 and 600 nm. In some embodiments, the composition has a turbidity comprised between 0 and 20 NTU.

Mention is made of the use of 1,3-butanediol to enhance or modulate the perceived olfactive impact and/or long-lastingness of a fragrance composition comprising a fragrance component and a volatile solvent with a vapor pressure above 0.1 mmHg at 20 °C in an amount not greater than 70%, typically 20% to 70%, by weight relative to the total weight of the fragrance composition.

The compositions, products, methods, and uses, according to the present disclosure are further illustrated by the following non-limiting examples.

### Example 1. Eau De Toilettes (EDTs) According to the Present Disclosure

Eau de Toilettes (EDTs) according to the present disclosure were made by combining a fragrance (“Fragrance A”) with the components and amounts summarized in Table 1 below. To obtain the compositions of the present disclosure, all components were weighed and mixed in a beaker under magnetic stirring. Fragrance, typically fragrance oil, was added to the mixture. Water was added and the solution was stirred until homogeneous.

TABLE 1

| | Control1 | EDT1 | EDT2 | EDT3 | EDT4 |
|---|---|---|---|---|---|
| Component | (wt %) | (wt %) | (wt %) | (wt %) | (wt %) |
| 1,3-butanediol | — | 13 | 5 | 10 | 5 |
| 1,2-propanediol¹⁾ | — | 13 | 13 | 8 | 12 |
| Hexyldecanol²⁾ | — | — | — | — | 1 |
| Fragrance A | 10 | 10 | 4 | 4 | 4 |
| Ethanol | 75 | 50 | 50 | 50 | 50 |
| Water | 15 | 14 | 28 | 28 | 28 |
| (demineralized) | | | | | |

¹⁾Radianol 4710; origin: Oleon
²⁾Eutanol G16; origin: BASF

“Fragrance A” had an inverted pyramid structure and contained the perfumery ingredients listed in Table 2 below.

TABLE 2
Fragrance A ingredients

decanal
octanal
1-[(2RS,3RS,8aRS)-2,3,8,8-tetramethyl-1,2,3,5,6,7,8,8a-octahydro-
2-naphthalenyl] ethanone
benzyl acetate
(−)-(5R)-5-isopropenyl-2-methyl-2-cyclohexen-1-one
(2Z)-3,7-dimethyl-2,6-octadienal
(+−)-3,7-dimethyl-6-octen-1-ol
(+−)-3-methyl-5-(2,2,3-trimethyl-3-cyclopenten-1-yl)-2-pentanol
ethyl (2E,4Z)-2,4-decadienoate
(3E,5Z)-1,3,5-undecatriene
lavender oil
1-methyl-4-(prop-1-en-2-yl)cyclohex-1-ene
(+−)-4-isopropenyl-1-methylcyclohexene
(−)-(3R)-3,7-dimethyl-1,6-octadien-3-ol
(+−)-3,7-dimethyl-1,6-octadien-3-yl acetate
methyl 2-(methylamino)benzoate
petitgrain oil
[2-[1-(3,3-dimethylcyclohexyl)ethoxy]-2-oxoethyl] propanoate
(1RS,2SR,4RS)-2-methoxy-4-propylcyclohexanol
dipropylene glycol

### Example 2. Sensory Panel

A sensory panel was performed in order to compare the reference EDT (“Control1”) and inventive EDT (“EDT1”), both of which contain Fragrance A, to measure olfactive performance.

A Prazitherm PZ72 slide warmer was pre-heated to 32 °C for 30 minutes. Blotters were placed on the precision hotplate. Using an adjustable volume pipette, 20 μl of solution was dosed directly to the center of the glass plate and evaporated at 32 °C.

At different times (t=0 min (Fresh), 2 hours, 4 hours and 6 hours), the randomized blotters were evaluated by 7 panelists.

The methodology used is a 3-Alternative Forced Choice test. For each time point, panelists were presented with 3 samples, two of which were the Reference EDT (“Control1”) and one of which was inventive EDT1.

Panelists indicated the sample(s) that they perceived higher in terms of overall intensity.

### Hypothesis:

H0: The two samples are not different.
H1: The sample with technology is more intense than the sample without technology, in terms of overall intensity.

### Associated Risks:

H0 rejected=α risk:
Risk associated with a false alarm, concluding that products differ when in fact they do not.
Data was analyzed using the binomial statistic.

### Data Interpretation:

If the p-value obtained for α≤0.05, then the sample with technology was more intense in overall intensity than the sample without technology,
If the p-value obtained for a is 0.05<α≤0.10, then a trend difference was determined,
If the p-value obtained for α>0.10, the samples were not significantly different.

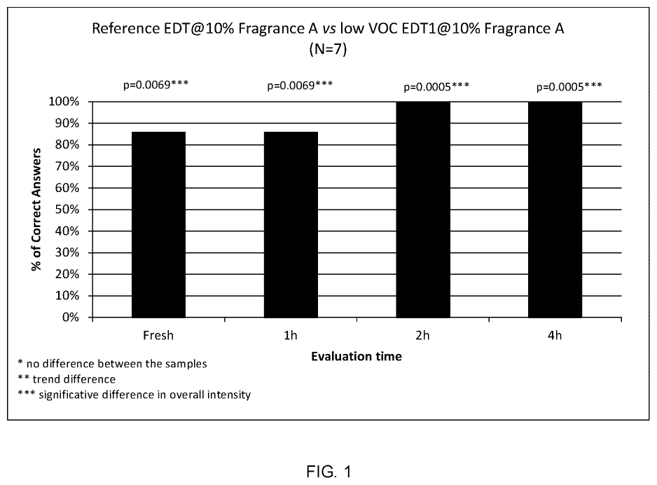

The result of the sensory evaluation is shown in FIG. 1. FIG. 1 shows a comparison of the olfactive performance between a reference EDT (“Control1”) and inventive EDT (“EDT1”), both of which contain the same fragrance. The results of the sensory panel presented in FIG. 1 show a significant higher performance of Fragrance A in EDT1 compared to the reference EDT at all time points. Therefore, it has been shown that the EDT1 provides more impact and long-lastingness to the fragrance compared to Reference EDT.

### Example 3. Influence of Fragrance Construction

In order to study the influence of fragrance construction, as characterized by the vapor pressure of the components of the fragrance ingredients, on impact and long-lastingness in the inventive EDTs, sensory panels were performed on several fragrances. The fragrances, characterized by percentage of ingredients having vapor pressure above and below 0.0008 Torr (at 25 °C) (Table 3), control formulations (Table 4), and inventive formulations (Table 5) are shown below.

TABLE 3

| Fragrance | Fragrance construction |
|---|---|
| ICE | 64% > 0.0008 Torr |
| | 36% < 0.0008 torr |
| SR | 54% > 0.0008 Torr |
| | 46% < 0.0008 torr |
| GG | 47% > 0.0008 Torr |
| | 53% < 0.0008 torr |
| LFB | 25% > 0.0008 Torr |
| | 75% < 0.0008 torr |

TABLE 4

| | Control2 | Control3 | Control4 | Control 5 |
|---|---|---|---|---|
| Component | (wt %) | (wt %) | (wt %) | (wt %) |
| Fragrance ICE | 10 | — | — | — |
| Fragrance SR | — | 10 | — | — |
| Fragrance GG | — | — | 10 | — |
| Fragrance LFB | — | — | — | 10 |
| Ethanol | 75 | 75 | 75 | 75 |
| Water | 15 | 15 | 15 | 15 |
| (demineralized) | | | | |

TABLE 5

| | EDT5 | EDT6 | EDT7 | EDT8 |
|---|---|---|---|---|
| Component | (wt %) | (wt %) | (wt %) | (wt %) |
| 1,3-butanediol | 13 | 13 | 13 | 13 |
| 1,2-propanediol | 13 | 13 | 13 | 13 |
| Fragrance ICE | 10 | — | — | — |
| Fragrance SR | — | 10 | — | — |
| Fragrance GG | — | — | 10 | — |
| Fragrance LFB | — | — | — | 10 |
| Ethanol | 50 | 50 | 50 | 50 |
| Water | 14 | 14 | 14 | 14 |
| (demineralized) | | | | |

The inventive EDTs in Table 5 were compared to their corresponding control formulation in Table 4 using the methodology described in Example 2.

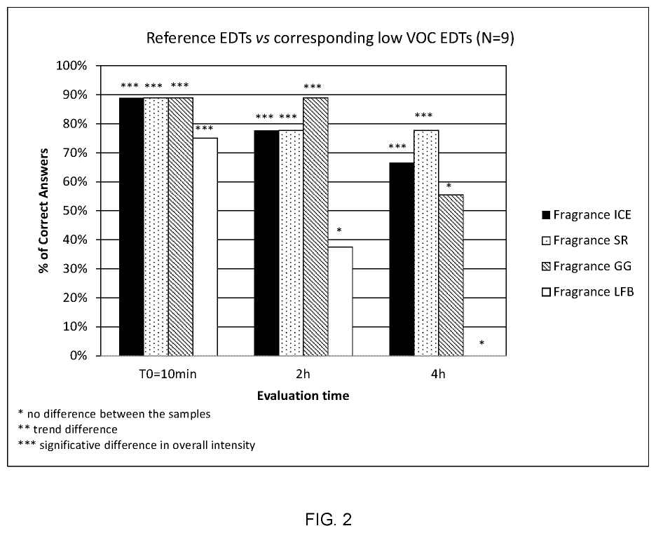

The results of the sensory panels are summarized in FIG. 2. FIG. 2 shows a comparison of the olfactive performance between the inventive EDTs in Table 5 as compared to their corresponding control formulation in Table 4. Table 6 below summarizes the p-values of the results shown in FIG. 2.

TABLE 6

| | | p- value | | |
|---|---|---|---|---|
| Fragrance | N | T0 | 2 hours | 4 hours |
| ICE | 9 | 0.0010 | 0.0083 | 0.0424 |
| SR | 9 | 0.0010 | 0.0083 | 0.0083 |
| GG | 9 | 0.0010 | 0.0010 | 0.1448 |
| LFB | 9 | 0.0197 | 0.5318 | 1.0000 |

According to these results, olfactive impact and long-lastingness was achieved by the inventive low VOC EDTs. It was found that olfactive impact and long-lastingness increased with the percentage of fragrance materials having a vapor pressure above 0.0008 Torr at 25 °C.

### Example 4. Influence of 1,3-butanediol Dosage

In order to study the influence of 1,3-butanediol dosage on fragrance impact, a sensory panel was performed to compare Fragrance SR in a low VOC formulation containing 5% of 1,3-butanediol and Fragrance SR in a low VOC formulation containing 10% of 1,3-butanediol. The two low VOC formulations are shown in Table 7 below.

TABLE 7

| | EDT9 | EDT10 |
|---|---|---|
| Component | (wt %) | (wt %) |
| 1,3-butanediol | 5 | 10 |
| 1,2-propanediol | 13 | 8 |
| Fragrance SR | 4 | 4 |
| Ethanol | 50 | 50 |
| Water | 28 | 28 |
| (demineralized) | | |

EDT9 and EDT10 were compared using the methodology described in Example 2, EDT9 being considered the reference.

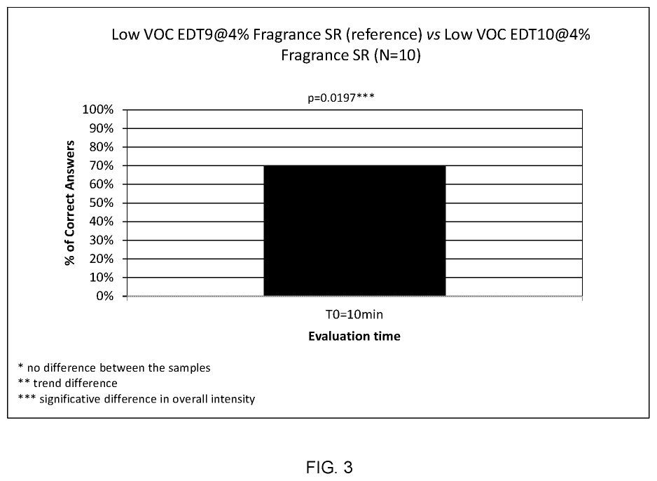

The results of the sensory panel are summarized in FIG. 3. FIG. 3 shows a comparison of the olfactive performance between EDT9 and EDT10. The results show a significantly higher performance of Fragrance SR in EDT10 compared to EDT9. Therefore, it has been shown that 10% 1,3-butanediol provides more impact to the Fragrance SR than 5% 1,3-butanediol.

### Example 5. Use of a Fragrance Modulator

In order to study the influence of addition of a fragrance modulator, or fixative, a sensory panel was performed to compare EDT9 according to Example 4 and Fragrance SR in a low VOC EDT (“EDT11”) containing 1% of a fragrance modulator, hexyldecanol, as shown in Table 8.

TABLE 8

| | EDT11 |
|---|---|
| Component | (wt %) |
| 1,3-butanediol | 5 |
| 1,2-propanediol | 12 |
| Hexyldecanol | 1 |
| Fragrance SR | 4 |
| Ethanol | 50 |
| Water | 28 |
| (demineralized) | |

EDT9 and EDT11 were compared using the methodology described in Example 2 in which EDT9 was considered the reference.

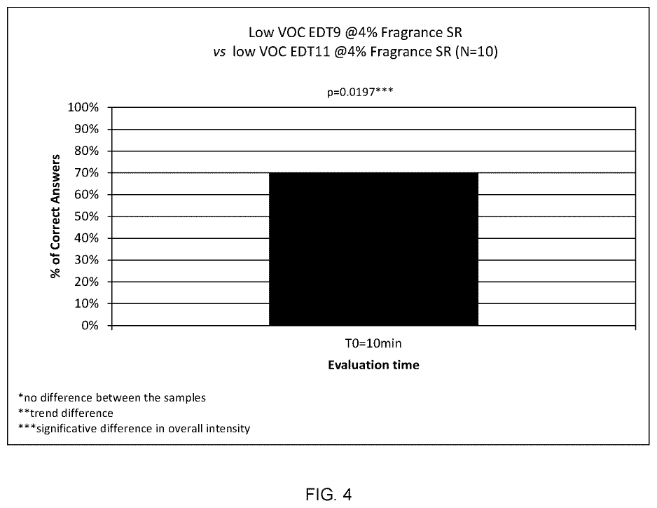

The results of the sensory panel are summarized in FIG. 4. FIG. 4 shows a comparison of the olfactive performance between EDT9 and EDT11. The results of the sensory panel show a significantly higher performance of Fragrance SR in EDT11 compared to EDT9, indicating that the addition of hexyldecanol provides greater impact to the Fragrance SR.

### Example 6. Solubility Test

This test was conducted on a fragrance (“Fragrance QS”) containing a high quantity of perfumery ingredients characterized with a log P above 5 and which would cause solubility issues in hydroalcoholic solutions due to high lipophilic content. Two low VOC formulations were prepared, as shown in Table 9 below.

TABLE 9

| | | EDT13 |
|---|---|---|
| Component | EDT12 | (wt %) |
| 1,3-butanediol | 5 | 5 |
| 1,2-propanediol | 13 | 12 |
| Hexyldecanol | — | 1 |
| Fragrance QS | 4 | 4 |
| Ethanol | 50 | 50 |
| Water | 28 | 28 |
| (demineralized) | | |

EDT12 and EDT13 were prepared in order to study the influence of the addition of hexyldecanol on solubility. The EDT formulations were placed under extreme conditions of temperature variations for 6 days: 3 cycles of 24 h @ −20 °C/24 h @ +25 °C At the end of the test, the aspect of each sample was evaluated.

A slight phase separation was observed in EDT12 whereas EDT13 was clear and homogeneous. Thus, it was shown that the addition of hexyldecanol improved solubility of lipophilic perfumery ingredients.

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those of ordinary skill in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein.

1. A fragrance composition comprising at least:
a) 1,3-butanediol,
b) a fragrance component, wherein the fragrance component comprises greater than 25% by weight of perfumery ingredients having a vapor pressure above 0.0008 Torr at 25 °C, relative to the weight of the fragrance component, and
c) a volatile solvent with a vapor pressure above 0.1 mmHg at 20 °C in an amount not greater than 70% by weight relative to the total weight of the fragrance composition.

2. The fragrance composition according to claim 1, wherein 1,3-butanediol is present in an amount of at least 1% by weight relative to the total weight of the fragrance composition.

3. The fragrance composition according to claim 1, wherein the fragrance component is present in an amount of less than 50% by weight relative to the total weight of the fragrance composition.

4. The fragrance composition according to claim 1, wherein the fragrance component comprises greater than 45% by weight of perfumery ingredients having a vapor pressure above 0.0008 Torr at 25 °C, relative to the weight of the fragrance component.

5. The fragrance composition according to claim 4, wherein the fragrance component comprises 45% to 99% by weight of perfumery ingredients having a vapor pressure above 0.0008 Torr at 25 °C, relative to the weight of the fragrance component.

6. The fragrance composition according to claim 1, further comprising at least one amphiphilic organic solvent different from 1,3-butanediol and characterized by a log P greater than or equal to −2 and less than or equal to 2.

7. The fragrance composition according to claim 6, wherein the at least one amphiphilic organic solvent is selected from the group consisting of glycols, dipropylene glycol, polyalkylene glycols, C₁-C₁₀ alkyl esters of citric acid, and combinations thereof.

8. The fragrance composition according to claim 6, wherein the at least one amphiphilic organic solvent is present in an amount of from 0.1% to 50% by weight relative to the total weight of the fragrance composition.

9. The fragrance composition according to claim 1, wherein the volatile solvent with a vapor pressure above 0.1 mmHg at 20 °C is in an amount not greater than 50% by weight relative to the total weight of the fragrance composition.

10. The fragrance composition according to claim 1, further comprising at least one fragrance modulator.

11. The fragrance composition according to claim 10, wherein the at least one fragrance modulator is hexyldecanol.

12. The fragrance composition according to claim 1, further comprising water.

13. The fragrance composition according to claim 12, wherein water is present in an amount of from 0.1% to 99% by weight relative to the total weight of the fragrance composition.

14. A consumer product comprising the fragrance composition according to claim 1.

15. The consumer product according to claim 14, wherein the consumer product is a perfume, a body-care product, a cosmetic preparation, a skin-care product, a fabric care product, an air care product, or a home care product.

16. The consumer product according to claim 14, wherein the consumer product is a fine perfume, a splash, an eau de toilette, an eau de parfum, a cologne, a body mist, a body spray, a hair mist, a shave or after-shave lotion, a shampoo, a coloring preparation, a color care product, a hair shaping product, a dental care product, a disinfectant, an intimate care product, a hair spray, a vanishing cream, a deodorant or antiperspirant, hair remover, tanning or sun product, a nail product, a skin cleansing, a makeup, a perfumed soap, a shower or bath mousse, oil or gel, a foot/hand care products, a hygiene product, a liquid or solid or unit-dose detergent, a fabric softener, a solid or liquid fabric scent-booster, a fabric refresher, an ironing water, an air freshener, a “ready to use” powdered air freshener, a mold remover, a furniture care product, a wipe, a dish detergent or hard-surface detergent, a leather care product, or a car care product.

17. A method for enhancing or modulating the perceived olfactive impact and/or long-lastingness of a fragrance composition, the method comprising combining 1,3-butanediol with a fragrance component, and a volatile solvent with a vapor pressure above 0.1 mmHg at 20 °C to obtain the fragrance composition, wherein the volatile solvent with a vapor pressure above 0.1 mmHg at 20 °C is in an amount not greater than 70% by weight relative to the total weight of the fragrance composition.

18. (canceled)

19. The fragrance composition according to claim 7, wherein the at least one amphiphilic organic solvent is selected from the group consisting of 1,2-alkanediols, 1,3-alkanediols, 1,4-alkanediols, and combinations thereof.

20. The fragrance composition according to claim 19, wherein the at least one amphiphilic organic solvent is selected from the group consisting of 1,2-propanediol, 1,2-butanediol, 1,2-pentanediol, 1,2-hexanediol, 1,2-heptanediol, 1,2-octanediol, and combinations thereof.
